(19) United States
(12) Patent Application Publication
(10) Pub. No.: US 2005/0181982 A1
(43) Pub. Date: Aug. 18, 2005

(54) Crystal structures of anti-factor IX Fab fragments and methods of use for peptidomimetic design

(75) Inventor: Manal A. Swairjo, San Diego, CA (US)

Correspondence Address: GLAXOSMITHKLINE;Corporate Intellectual Property - UW2220, P.O. Box 1539, King of Prussia, PA, 19406-0939, US

(73) Assignee: SmithKline Beecham Corporation

(21) Appl. No.: 11/104,756

(22) Filed: Apr. 13, 2005

Related U.S. Application Data
(63) Continuation of application No. 10/144,602, filed on May 13, 2002.
Application No. 10/144,602 is a continuation of application No. 09/669,910, filed on Sep. 26, 2000, now abandoned.
Application No. 09/669,910 is a continuation of application No. 09/108,131, filed on Jun. 30, 1998, now abandoned.
(60) Provisional application No. 60/051,645, filed on Jul. 3, 1997.

(51) Int. Cl. G06F 19/00; G01N 33/48; G01N 33/50

(52) U.S. Cl. 514/2; 530/350; 702/19; 530/383

(57) ABSTRACT

Novel anti-Factor IX Fab fragment crystalline structures are identified. Methods of identifying peptidomimetics of these fragments are disclosed

This application claims the benefit of U.S. Provisional Application No. 60/051,645, filed 3 Jul. 1997.

### FIELD OF THE INVENTION

This invention relates to anti-Factor IX Fab fragment crystals and the use of complementarity determining region (CDR) structural parameters for design and selection of peptidomimetics.

### BACKGROUND OF THE INVENTION

Under normal circumstances, an injury, be it minor or major, to vascular endothelial cells lining a blood vessel triggers a hemostatic response through a sequence of events commonly referred to as the coagulation “cascade.” The cascade culminates in the conversion of soluble fibrinogen to insoluble fibrin which, together with platelets, forms a localized clot or thrombus which prevents extravasation of blood components. Wound healing can then occur followed by clot dissolution and restoration of blood vessel integrity and flow.

The events which occur between injury and clot formation are a carefully regulated and linked series of reactions. In brief, a number of plasma coagulation proteins in inactive proenzyme forms and cofactors circulate in the blood. Active enzyme complexes are assembled at an injury site and are sequentially activated to serine proteases, with each successive serine protease catalyzing the subsequent proenzyme to protease activation. This enzymatic cascade results in each step magnifying the effect of the succeeding step. For an overview of the coagulation cascade see the first chapter of “Thombosis and Hemorrhage”, J. Loscalzo and A. Schafer, eds., Blackwell Scientific Publications, Oxford, England (1994).

While efficient clotting limits the loss of blood at an injury site, inappropriate formation of thrombi in veins or arteries is a common cause of disability and death. Abnormal clotting activity can result in and/or from pathologies or treatments such as myocardial infarction, unstable angina, atrial fibrillation, stroke, renal damage, percutaneous translumenal coronary angioplasty, disseminated intravascular coagulation, sepsis, pulmonary embolism and deep vein thrombosis. The formation of clots on foreign surfaces of artificial organs, shunts and prostheses such as artificial heart valves is also problematic.

Approved anticoagulant agents currently used in treatment of these pathologies and other thrombotic and embolic disorders include the sulfated heteropolysaccharides heparin and low molecular weight (LMW) heparin. These agents are administered parenterally and can cause rapid and complete inhibition of clotting by activation of the thrombin inhibitor, antithrombin III and inactivation of all of the clotting factors.

However, due to their potency, heparin and LMW heparin suffer drawbacks. Uncontrolled bleeding as a result of the simple stresses of motion and accompanying contacts with physical objects or at surgical sites is the major complication and is observed in 1 to 7% of patients receiving continuous infusion and in 8 to 14% of patients given intermittent bolus doses. To minimize this risk, samples are continuously drawn to enable ex vivo clotting times to be continuously monitored, which contributes substantially to the cost of therapy and the patient's inconvenience.

Further, the therapeutic target range to achieve the desired level of efficacy without placing the patient at risk for bleeding is narrow. The therapeutic range is approximately 1 to less than 3 ug heparin/ml plasma which results in activated partial thromboplastin time (aPTT) assay times of about 35 to about 100 seconds. Increasing the heparin concentration to 3 ug/ml exceeds the target range and at concentrations greater than 4 ug/ml, clotting activity is not detectable. Thus, great care must be taken to keep the patient's plasma concentrations within the therapeutic range.

Another approved anticoagulant with slower and longer lasting effect is warfarin, a coumarin derivative. Warfarin acts by competing with Vitamin K dependent post-translational modification of prothrombin and other Vitamin K-dependent clotting factors.

The general pattern of anticoagulant action, in which blood is rendered non-clottable at concentrations only slightly higher than the therapeutic range is seen for warfarin as well as for heparin and LMW heparin. Clearly, a need exists for an anticoagulant agent which is efficacious in controlling thrombotic and embolic disorders yet does not cause uncontrolled bleeding or its possibility. Accordingly, there is also a need for anticoagulant agent structural information to enable identification and structure-based design of new anticoagulant agents.

### SUMMARY OF THE INVENTION

Accordingly, an aspect of the present invention is a BC2 Fab fragment crystal.

Another aspect of the invention is a Fab fragment crystal containing BC2 CDRs.

Another aspect of the invention is a SB249417 Fab fragment crystal.

Another aspect of the invention is a method for identifying a peptidomimetics having Factor IX binding activity comprising the steps of searching a small molecule structural database with CDR structural parameters derived from anti-Factor IX Fab fragment crystals: selecting a molecular structure from the database which mimics the CDR structural parameters: synthesizing the selected molecular structure; and screening the synthesized molecule for Factor IX binding activity.

### BRIEF DESCRIPTION OF THE DRAWINGS

FIG. 1 is a three-dimensional structure of the residues of BC2 HC-CDR1.

FIG. 2 is a three-dimensional structure of the residues of BC2 HC-CDR2.

FIG. 3 is a three-dimensional structure of the residues of BC2 HC-CDR3.

FIG. 4 is a three-dimensional structure of the residues of BC2 LC-CDR1.

FIG. 5 is a three-dimensional structure of the residues of BC2 LC-CDR2.

FIG. 6 is a three-dimensional structure of the residues of BC2 LC-CDR3.

FIG. 7 is a three-dimensional structure of the residues of SB249417 HC-CDR1.

FIG. 8 is a three-dimensional structure of the residues of SB249417 HC-CDR2.

FIG. 9 is a three-dimensional structure of the residues of SB249417 HC-CDR3.

FIG. 10 is a three-dimensional structure of the residues of SB249417 LC-CDR1.

FIG. 11 is a three-dimensional structure of the residues of SB249417 LC-CDR2.

FIG. 12 is a three-dimensional structure of the residues of SB249417 LC-CDR3.

### DETAILED DESCRIPTION OF THE INVENTION

All publications, including but not limited to patents and patent applications, cited in this specification are herein incorporated by reference as though fully set forth.

Factor IX (fIX) is a vitamin K-dependent serine protease zymogen which plays an important role in the amplification of the blood coagulation cascade by catalyzing the activation of factor X on the membrane surface in the presence of activated factor VIII and calcium. Murine anti-human factor IX monoclonal antibody (in Ab) BC2, as described in U.S. patent application Ser. No. 08/783,853 is an IgG1 kappa monoclonal antibody having useful properties for anticoagulant therapy in arterial and venous thrombosis. BC2 down-regulates the blood clotting cascade in a self-limiting manner. BC2 inhibits the activation of fIX to fIXa by fXI as well as its activation by the complex of tissue factor and fVIIa. BC2 also inhibits fIXa coagulant activity. BC2 binds to human fIX and fIXa in a calcium-dependent manner with a dissociation constant Kd=4 nM. BC2 also cross-reacts with and inhibits rat fIX.

Humanized constructs of BC2 have been made and tested for anticoagulant activity in vitro and in animal models. These constructs are described in U.S. patent application Ser. No. 08/783,853 and, like BC2, are novel anticoagulants exhibiting self-limiting, neutralizing activity, namely they down-regulate the blood clotting cascade in a self-limiting manner, minimizing the bleeding risks associated with heparin and other anticoagulant therapies. One such humanized construct of BC2 is SB249417. As used herein, the term “self-limiting, neutralizing activity” refers to the activity of a peptidomimetic that binds to human coagulation factor IX or IXa and inhibits thrombosis in a manner such that limited modulation of coagulation is produced. “Limited modulation of coagulation” is defined as an increase in clotting time, as measured by prolongation of the activated partial thromboplastin time (aPTT), where plasma remains clottable with aPTT reaching a maximal value despite increasing concentrations of monoclonal antibody. This limited modulation of coagulation is in contrast to plasma being rendered unclottable and exhibiting an infinite aPTT in the presence of increasing concentrations of heparin. Preferably, the maximal aPTT values are within the heparin therapeutic range. Most preferably, maximal aPTT is within the range of about 35 seconds to about 100 seconds which corresponds to about 1.5 times to about 3.5 times the normal control aPTT value.

In the humanization process, the mouse antibody framework is changed to that from a human antibody, leaving the antigen-binding site unchanged. This site is formed by certain regions in the mAb amino acid sequence which are termed the complementarity determining regions (CDRs), or hypervariable segments. The antigen-binding site, which determines its specificity to its antigen, is located in the Fab fragment of the antibody, which consists of the entire light chain (LC) and part of the heavy chain (HC). As part of an effort to develop functional small-molecule mimics of these therapeutic macromolecules, the structural and mechanistic features of the anticoagulant activity of the anti-fIX mAbs BC2 and SB249417 have been determined. This information is useful for design and testing of small peptides that functionally mimic the mAb's anticoagulant properties and to develop these peptides for therapeutic use.

The three-dimensional structures of the Fab fragments of BC2 and SB249417 were determined using X-ray crystallography as described in the Examples. The structural information can be stored on a computer-readable medium.

The CDRs from the mouse and humanized Fab fragments have generally similar conformations. R.m.s. differences between corresponding CDR C_{α} positions between the two Fabs are below 0.5 Å, except in HC-CDR2 and HC-CDR3 where r.m.s. values are 1.97 and 3.7 Å, respectively. The slight change in the conformations of HC-CDR2 and HC-CDR3 amount to an angular shift in the planes of these loops, keeping the angle between them unchanged. In both Fabs, the three HC CDRs and LC-CDR3 form a groove (27 Å long, 8 Å wide and 9 Å deep) which runs through the CDR surface. CDR residues HC-Asn35, HC-Trp50, and LC-Arg95, which line a deep hole in the center of the groove, are considered important for antigen binding.

Structural information obtained for the CDRs of the BC2 and SB249417 Fab structures is useful for discovery of small molecule peptidomimetics. Preferred peptidomimetics include peptides and synthetic organic molecules which bind to Factor IX and have self-limiting, neutralizing activity in an in vitro clotting assay. An exemplary approach to such a structure-based peptide mimic design follows (Zhao, et al., 1995; Monfardini C. et al., 1996).

A search of several small-molecule structural data bases such as Available Chemicals Directory, Cambridge Crystallographic Database, Fine Chemical Database and CONCORD database (for a review, see Rusinko A., 1993) is carried out using parameters derived from the CDR structures. The search can be 2-dimensional, 3-dimensional or both and can be done using a combination of software such as UNITY version 2.3.1 (Tripos, Inc.), MACCS 3D, CAVEAT and DOCK. Conformational flexibility of the small molecules is allowed. The strategy for conducting the search takes into account conformations of individual CDRs as well as combinations of CDRs and/or key residues in the mAb combining site.

An initial approach is to focus on structural parameters from HC-CDR3, LC-CDR3 and HC-CDR2 since these CDRs have been found in other Fabs to participate intimately in antigen recognition. A search for small-molecule mimics of HC-CDR3, LC-CDR3 and HC-CDR2 is separately conducted. The structural parameters from each two of these three CDRs are combined and the search repeated. The next step will be using parameters from all three CDRs. The conformational parameters of the remaining three CDRs will be included at a later stage, resulting in a search combining all six CDRs. Preferably, the selected molecular structure mimics the parameters of CDR residues HC-Asn35, HC-Trp50, and LC-Arg95. Small-molecule hits resulting from the searches are synthesized and screened for factor-IX binding in an ELISA assay and preferably, for anti-thrombotic activity in a standard in vitro clotting assay. Most preferably, the hits will also exhibit self-limiting, neutralizing activity.

Peptidomimetics produced by the method of the invention are expected to be useful in therapy of thrombotic and embolic disorders such as those associated with myocardial infarction, unstable angina, atrial fibrillation, stroke, renal damage, pulmonary embolism, deep vein thrombosis, percutaneous translumenal coronary angioplasty, disseminated intravascular coagulation, sepsis, artificial organs, shunts or prostheses.

The present invention will now be described with reference to the following specific, non-limiting examples.

### EXAMPLE 1

### Preparation and Purification of Fab Fragments

Both BC2 and SB249417 Fab fragments were prepared and purified as follows, 50 mL of freshly purified monoclonal anti-human fIX antibody sample (1.2 mg/mL in PBS buffer) was concentrated in an Amicon cell using a 30-kDa molecular weight cutoff membrane (YM30, at 65 psi, 4° C.) to a final volume of 5.0 mL and final concentration of 12.0 mg/mL. A papain digest of the mAb was started by adding to the concentrated mAb sample 20 μg/mL papain (Boehringer Manheim, cat.# 108014), 2.5 mM EDTA (pH 7.5) and 5.0 mM cysteine-HCL monohydrate (PIERCE, cat.# 44889) and incubating the mixture at 37° C. for 4 hours and shaking gently. The reaction was stopped by cooling the mixture on ice for 20 min.

The Fc fragment was removed by incubating the digest with 5 mL of protein A-Sepharose resin (Pharmacia) and mixing at 4° C. for 1 hour. The mixture was transferred into a 15 mL gravity-fed column, and the unbound fraction (containing the Fab fragment) was collected. The column was washed twice with a 8 mL volume of 20 mM Na₂HPO₄, 150 mM NaCl, pH 7.5. The eluate and 2 washes were pooled and concentrated to 5.3 mL using an Amicon cell with a YM10 membrane at 4° C.

The sample was loaded on a Pharmacia Superdex 75 column (volume 320 mL), pre-equilibrated with 20 nM Na₂HPO₄, 150 mM NaCl, pH 7.5. The column was then eluted with the same buffer at a rate of 2.5 mL/min, and 1 mL fractions collected after 30 min of void-volume collection. The Fab fragment eluted as a single molecular species as indicated by a large A₂₈₀ peak appearing in fractions 26-36, which were pooled and assayed for protein concentration by A₂₈₀ absorption. A total of 25 mg of Fab were generated using this standard protocol (purification yield=50-60%). SDS-PAGE analysis of the Superdex 75 eluate revealed a single species with an apparent molecular weight of 47,000 Da.

IEF analysis of the BC2 Fab sample revealed the presence of multiple isoelectric variants; the two major isoforms have apparent pI values of 8.9 and 7.35. These two species were separated using an ion exchange chromatography step which proved necessary and sufficient for obtaining usable crystals. The 25 mg SEC eluate was buffer exchanged by thorough and repeated dialysis against 20 mM Tris, pH 9.2, concentrated to 5 mL in an Amicon cell, and loaded on a 1 mL Pharmacia Mono Q column, pre-equilibrated with buffer A (20 mM Tris, pH 9.2). The column was washed with 10 mL buffer A, and no protein eluted in the flow through. Three protein species were eluted with a 0-15% gradient of buffer B (20 mM Tris, pH 9.2, 1.0M NaCl) followed by a 15-100% gradient of buffer B, at a rate of 1.0 mL/min. 1 mL fractions were collected. Fractions corresponding to the first (sharp) peak in the chromatogram were pooled, assayed for A₂₈₀ absorption, buffer exchanged in an Amicon cell against 20 mM HEPES, pH 7.4, concentrated to 5 mg/mL and used for crystallization. Fractions from the other two peaks did not crystallize. The final yield of the protocol was approximately 36% (crystallizable fraction only).

### EXAMPLE 2

### Crystallization of Fab Fragments

BC2 Fab: Protein isoform from peak 1 of the ion exchange step was crystallized using the vapor diffusion method in a sitting-drop setup. The well solution contained 14% PEG6K, 20 mM ammonium sulfate (or 100 mM LiCl), 10 mM CaAc₂ and 200 mM imidazole/HEPES, pH 7.0. The drops were prepared by mixing 3 μL of the well solution with 3 μL of protein solution (8 mg/mL in 20 mM HEPES, pH 7.0). Large orthorhombic crystals grew in 5 days at 21° C. to a size of 0.8×0.3×0.25 min³. The crystals diffracted to 3.0 Å, in space group P21212, unit cell dimensions a=89.3, b=120.6, c=43.4 Å, and one molecule in the asymmetric unit.

SB249417 Fab: A similar sitting drop method was used. The well solution contained 30-40% saturated ammonium sulfate and 50 mM MES, pH 6.0. The drops were prepared by mixing equal volumes of well solution and protein solution (10 mg/mL in 10 mM HEPES, pH 7.0). Large crystals grew in one week at 15° C. to a size of 0.6×0.4×0.3 mm³. The crystals diffracted to 2.2 Å, in space group P1, unit cell dimensions a=56.6, b=56.6, c=73.7 Å, α=86.0, β=86.0, γ=64.9°, and two molecules in the asymmetric unit.

### EXAMPLE 3

### X-Ray Data Collection

X-ray diffraction data were collected on a MAR area detector mounted on a Rigaku high-brilliance source operated at 50 kV/100 mA with monochromatic CuK_{α} radiation in 1° oscillations frames. Data from three and two different crystals were collected, merged and used for structure determination of the BC2 Fab and SB249417 Fab, respectively. All data were processed using the HKL program, edition 4 (Otwinowski, 1993). Table 1 summarizes the data collection parameters.

For BC2, the merged data were used for structure determination, whereas structure refinement was done against a single-crystal data set with the best R-sym values. For SB249417, merged data were used for structure determination and refinement.

TABLE 1
Summary of X-ray Diffraction Data.

| Parameter | BC2 | 249417 |
|---|---|---|
| cell a, b, c (Å) | 89.60, 120.69, 43.58 | 56.6, 56.6, 73.7 |
| alpha, beta, gamma | 90.0, 90.0, 90.0 deg. | 86.0, 86.0, 64.9 deg. |
| Resolution (Å) | 3.0 | 2.2 |
| Number of | 132,951 | 145,877 |
| observed reflections | | |
| Number of | 12,211 | 21,122 |
| unique reflections | | |
| mosaicity | 0.16 | 0.22 |
| <I/σ> | 11.5 | 7.0 |
| Completeness | 99.7 | 99.9 |
| % of data >2σ | 76.0 | 71.4 |
| R-sym | 0.12 | 0.07 |

### EXAMPLE 4

### Structure Determination

The structures of the Fabs were determined using generalized molecular replacement methods following the standard protocol of Brünger (1991). The procedure includes a real-space cross-rotation Patterson search (Huber, 1985) followed by Patterson coefficient (PC) refinement (Brünger, 1990), a translation search, and finally rigid-body refinement. The X-PLOR program suite was used (Brünger, 1992) for all four steps.

A search model was constructed for BC2 from the PDB-deposited 1.9 Å structures of two Fabs: the light chain model from murine IgG2a Fab that neutralizes human rhinovirus 14 (PDB entry 1FOR), and the heavy chain model from murine idiotype Fab 730.1.4 (PDB entry 1IAI). The two were combined by least-square fitting of the two-chain models. Sequence identity of the resulting probe with BC2 Fab is as follows:
V_{L} 84%
C_{L} 100%
V_{H} 84%
C_{H}1 95%,

A similar search model was constructed for SB249417 from the PDB-deposited 3.0 Å humanized anti-CD 18 antibody Fab fragment (PDB entry 2FGW). Sequence identity of the search model with SB249417 Fab is as follows:
V_{L} 81%
C_{L} 100%
V_{H} 59%
C_{H}1 99%

In each model, residues different from those in the amino acid sequence of the Fab were mutated to alanine.

In the case of BC2, a cross-rotation search was done with this model which represents the entire asymmetric unit. Eulerian space was searched in the rotation-function's asymmetric unit (0≦θ₁<2, 0≦θ₂≦/2, 0≦θ₃<, where θ₁, θ₂, θ₃ are the Eulerian angles as defined by Rossmann & Blow (1962)) with a constant increment of 2.5° in each dimension. Data in the resolution range 15.0-4.0 Å was used in this search. The top 6000 peaks of the rotation function (RF) were used for cluster analysis. The solutions of the rotation function were then subjected to PC refinement followed by rigid-body minimization of the solution with the highest PC value. The latter was done in three steps: 1) treating the entire molecular model as a rigid body, 2) treating the heavy chain and light chain each as a rigid body and 3) treating the variable (V_{H} and V_{L}) and constant (C_{H}1 and C_{L}) domains of each chain as a rigid body.

In the case of SB249417, an initial self-rotation search converged to a single solution representing a non-crystallographic two-fold axis defined by spherical angles psi, phi=147, 0. A cross-rotation search (0≦θ₁<2, 0≦θ₂≦, 0≦θ₃<2) was followed by PC refinement, resulting in two solutions, which were related by non-crystallographic symmetry.

Using the structure corresponding to the highest RF peak after PC refinement (one peak in the case of BC2 and two peaks related by NCS in the case of SB249417) and 15.0-4.0 Å data, a translation search was carried out. For BC2, the search was restricted to half of the unit cell in all three dimensions. For SB249417, NCS was directly applied to the translation function solution to generate the other molecule in the P1 cell. For each Fab, the structure corresponding to the top solution of the translation function was then rigid-body refined as described above.

The rigid-body refined structure was then used to phase the reflections from a single-crystal data set, in the case of BC2, or merged data from multiple crystals in the case of SB249417. Fₒ-F_{c} and 2Fₒ-F_{c} electron density maps were calculated and inspected. The model was re-built to fit the map in the CDR regions and elsewhere using the true amino acid sequence of the Fab. The structures were refined using the simulated annealing protocols of X-PLOR (Brünger, 1992). Refinement parameters are summarized in Table 2.

TABLE 2
Structure Refinement Statistics

| Parameter | BC2 | SB249417 |
|---|---|---|
| Space group | P2₁ 2₁ 2 | P1 |
| Observations (N) | 47,643 | 145,877 |
| Unique reflections (N) | 11,353 | 40,746 |
| R-sym (on I, %) | 0.09 | 0.07 |
| Average I/s | 8.8 | 7.1 |
| Reflections use in refinement (N) | 8469 | 36,628 |
| Completeness of refinement data | 92.2 | 94.3 |
| Refinement resolution range (Å) | 20.0-3.0 | 15.0-2.2 |
| Atoms used in refinement (N) | 3157 | 6481 |
| R_{cryst} (%) | 22.0 | 23.0 |
| R_{free} (%) | 29.0 | 27.9 |
| R.m.s. deviations from | | |
| standard values: | | |
| Bond length (Å) | 0.019 | 0.014 |
| Bond angles (deg.) | 3.3 | 1.27 |
| Mean B-factor (Å²) | 29.0 | 27.3 |

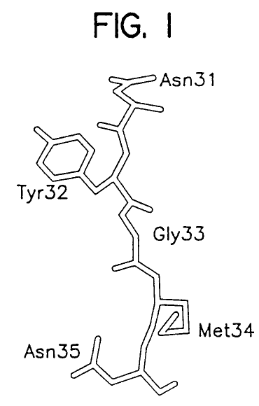

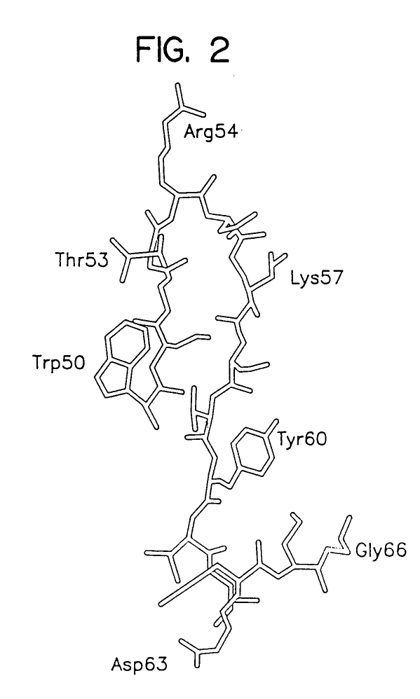

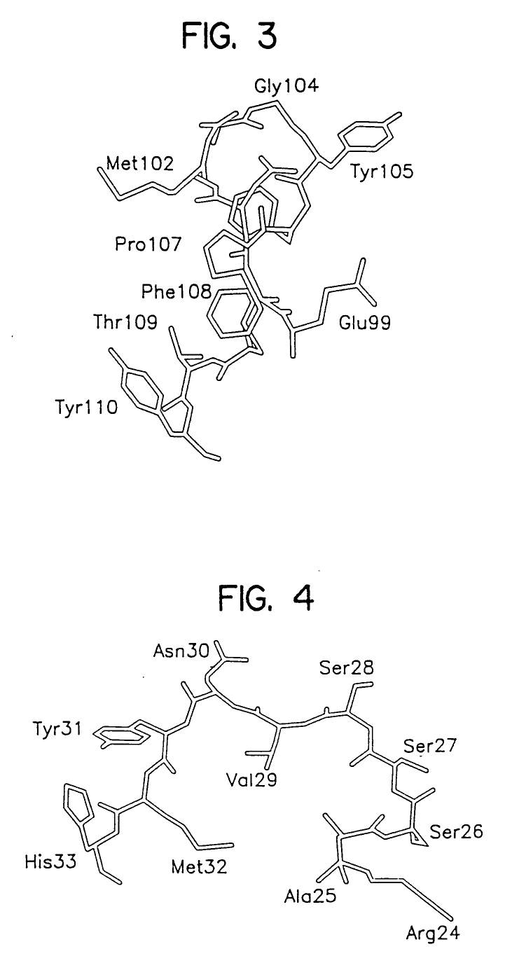

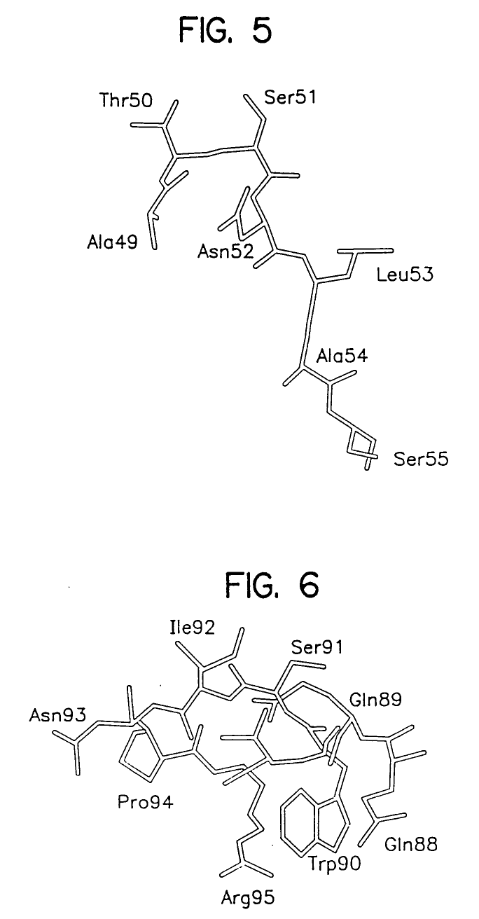

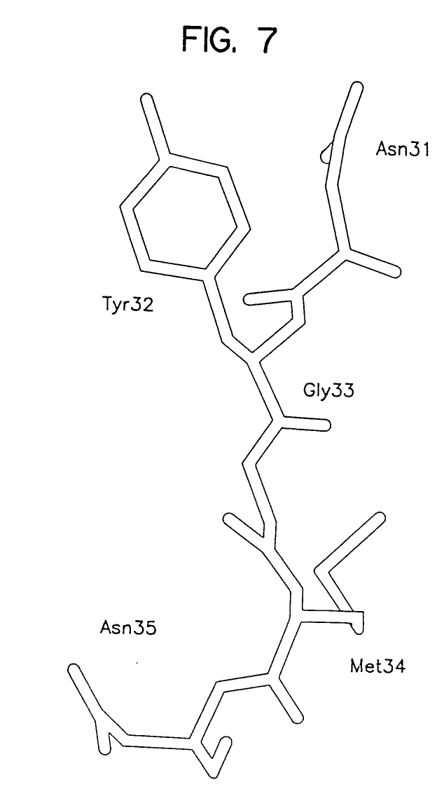

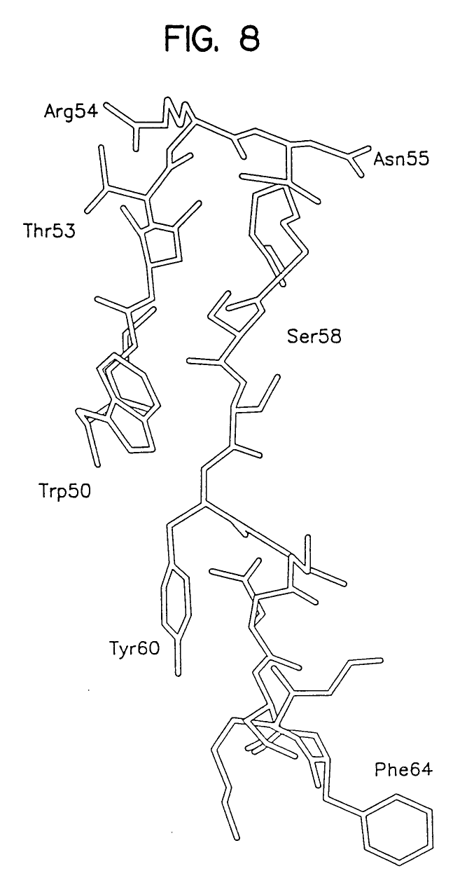

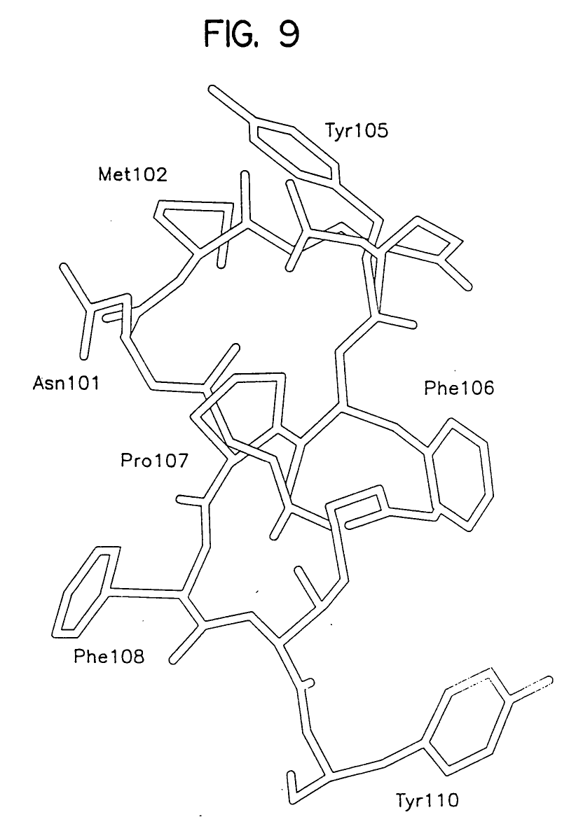

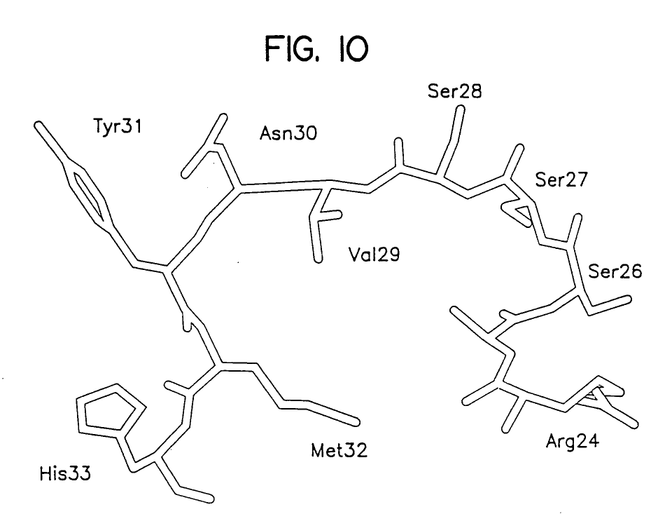

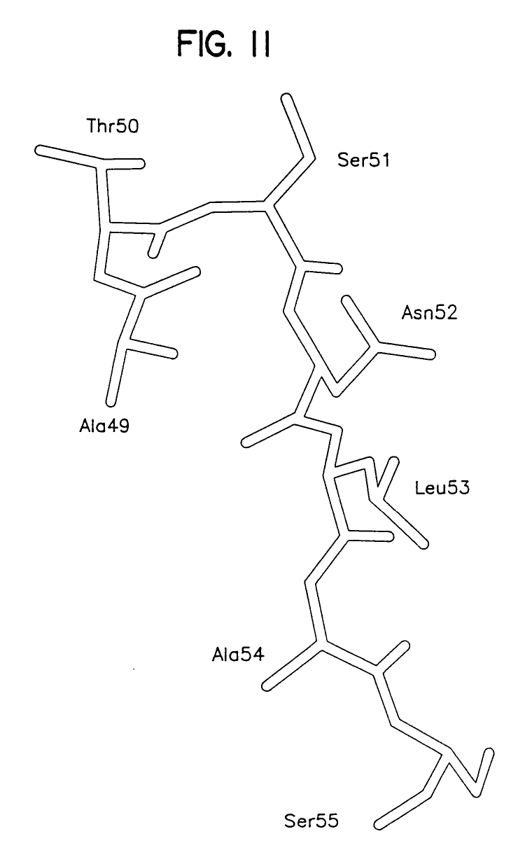

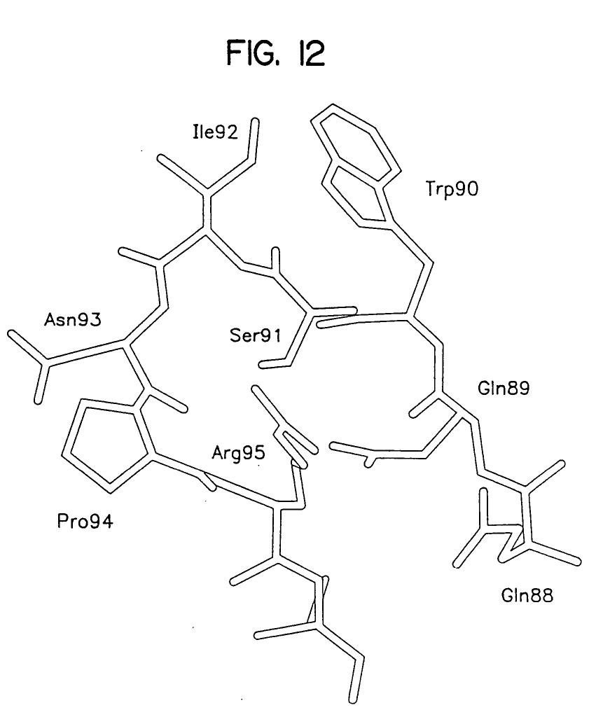

Like all Fab fragments, BC2 and SB249417 Fab structures are made up of a tetrahedral array of four globular domains—V_{L}, V_{H}, C_{L} and C_{H}1—which follow the immunoglobulin fold. Each domain is constituted of two broad sheets of antiparallel β-strands held together by hydrophobic interactions. The CDR loops are ordered with varying temperature-factor values. The three-dimensional coordinates of the residues belonging to all six CDRs of BC2 and SB249417 are listed in Tables 3-8 and Tables 9-14, respectively. FIGS. 1-6 and 7-12 show the corresponding three dimensional structures.

TABLE 3
Three dimensional coordinates of
HC - CDR1 (HC: ASN31-ASN35) from BC2

| | | | | | x | y | z | Q | B |
|---|---|---|---|---|---|---|---|---|---|
| ATOM | 2287 | N | ASN | 31 | 38.145 | 52.427 | −13.427 | 1.00 | 48.47 |
| ATOM | 2289 | CA | ASN | 31 | 37.357 | 53.503 | −12.856 | 1.00 | 48.47 |
| ATOM | 2290 | CB | ASN | 31 | 35.961 | 53.611 | −13.477 | 1.00 | 49.47 |
| ATOM | 2291 | CG | ASN | 31 | 35.742 | 52.671 | −14.676 | 1.00 | 49.47 |
| ATOM | 2292 | OD1 | ASN | 31 | 36.684 | 52.260 | −15.365 | 1.00 | 49.47 |
| ATOM | 2293 | ND2 | ASN | 31 | 34.477 | 52.330 | −14.916 | 1.00 | 49.47 |
| ATOM | 2296 | C | ASN | 31 | 37.231 | 53.540 | −11.325 | 1.00 | 48.47 |
| ATOM | 2297 | O | ASN | 31 | 36.898 | 54.595 | −10.776 | 1.00 | 49.47 |
| ATOM | 2298 | N | TYR | 32 | 37.491 | 52.428 | −10.636 | 1.00 | 55.29 |
| ATOM | 2300 | CA | TYR | 32 | 37.341 | 52.392 | −9.167 | 1.00 | 55.29 |
| ATOM | 2301 | CB | TYR | 32 | 36.051 | 51.709 | −8.737 | 1.00 | 25.46 |
| ATOM | 2302 | CG | TYR | 32 | 34.839 | 51.959 | −9.549 | 1.00 | 25.46 |
| ATOM | 2303 | CD1 | TYR | 32 | 34.842 | 51.790 | −10.936 | 1.00 | 25.46 |
| ATOM | 2304 | CE1 | TYR | 32 | 33.672 | 51.848 | −11.656 | 1.00 | 25.46 |
| ATOM | 2305 | CD2 | TYR | 32 | 33.642 | 52.198 | −8.911 | 1.00 | 25.46 |
| ATOM | 2306 | CE2 | TYR | 32 | 32.466 | 52.244 | −9.600 | 1.00 | 25.46 |
| ATOM | 2307 | CZ | TYR | 32 | 32.475 | 52.071 | −10.966 | 1.00 | 25.46 |
| ATOM | 2308 | OH | TYR | 32 | 31.269 | 52.059 | −11.601 | 1.00 | 25.46 |
| ATOM | 2310 | C | TYR | 32 | 38.442 | 51.679 | −8.402 | 1.00 | 55.29 |
| ATOM | 2311 | O | TYR | 32 | 38.845 | 50.570 | −8.772 | 1.00 | 25.45 |
| ATOM | 2312 | N | GLY | 33 | 38.774 | 52.229 | −7.237 | 1.00 | 17.19 |
| ATOM | 2314 | CA | GLY | 33 | 39.817 | 51.656 | −6.405 | 1.00 | 17.19 |
| ATOM | 2315 | C | GLY | 33 | 39.406 | 50.378 | −5.697 | 1.00 | 17.19 |
| ATOM | 2316 | O | GLY | 33 | 38.237 | 50.200 | −5.296 | 1.00 | 65.52 |
| ATOM | 2317 | N | MET | 34 | 40.382 | 49.487 | −5.526 | 1.00 | 36.25 |
| ATOM | 2319 | CA | MET | 34 | 40.143 | 48.215 | −4.854 | 1.00 | 36.25 |
| ATOM | 2320 | CB | MET | 34 | 40.888 | 47.087 | −5.555 | 1.00 | 15.05 |
| ATOM | 2321 | CG | MET | 34 | 40.667 | 45.723 | −4.926 | 1.00 | 15.05 |
| ATOM | 2322 | SD | MET | 34 | 38.944 | 45.396 | −4.815 | 1.00 | 15.05 |
| ATOM | 2323 | CE | MET | 34 | 38.703 | 44.674 | −6.413 | 1.00 | 15.05 |
| ATOM | 2324 | C | MET | 34 | 40.635 | 48.287 | −3.430 | 1.00 | 36.25 |
| ATOM | 2325 | O | MET | 34 | 41.514 | 49.072 | −3.107 | 1.00 | 15.05 |
| ATOM | 2326 | N | ASN | 35 | 40.072 | 47.454 | −2.570 | 1.00 | 16.44 |
| ATOM | 2328 | CA | ASN | 35 | 40.513 | 47.391 | −1.182 | 1.00 | 16.44 |
| ATOM | 2329 | CB | ASN | 35 | 39.359 | 47.668 | −0.196 | 1.00 | 23.13 |
| ATOM | 2330 | CG | ASN | 35 | 38.947 | 49.118 | −0.149 | 1.00 | 23.13 |
| ATOM | 2331 | OD1 | ASN | 35 | 38.491 | 49.623 | −0.888 | 1.00 | 23.13 |
| ATOM | 2332 | ND2 | ASN | 35 | 39.065 | 49.793 | −1.275 | 1.00 | 23.13 |
| ATOM | 2335 | C | ASN | 35 | 41.038 | 45.954 | −0.980 | 1.00 | 16.44 |
| ATOM | 2336 | O | ASN | 35 | 41.058 | 45.182 | −1.920 | 1.00 | 23.13 |

TABLE 4
Three dimensional coordinates of
HC - CDR2 (HC: TRP50-GLY66) from BC2

| | | | | | x | y | z | Q | B |
|---|---|---|---|---|---|---|---|---|---|
| ATOM | 2474 | N | TRP | 50 | 45.028 | 49.852 | −0.044 | 1.00 | 2.00 |
| ATOM | 2476 | CA | TRP | 50 | 44.159 | 50.501 | −1.002 | 1.00 | 2.00 |
| ATOM | 2477 | CB | TRP | 50 | 44.044 | 51.944 | −0.556 | 1.00 | 57.49 |
| ATOM | 2478 | CG | TRP | 50 | 42.874 | 52.695 | −1.042 | 1.00 | 57.49 |
| ATOM | 2479 | CD2 | TRP | 50 | 42.803 | 53.588 | −2.163 | 1.00 | 57.49 |
| ATOM | 2480 | CE2 | TRP | 50 | 41.556 | 54.226 | −2.120 | 1.00 | 57.49 |
| ATOM | 2481 | CE3 | TRP | 50 | 43.669 | 53.919 | −3.196 | 1.00 | 57.49 |
| ATOM | 2482 | CD1 | TRP | 50 | 41.703 | 52.803 | −0.412 | 1.00 | 57.49 |
| ATOM | 2483 | NE1 | TRP | 50 | 40.904 | 53.723 | −1.037 | 1.00 | 57.49 |
| ATOM | 2485 | CZ2 | TRP | 50 | 41.155 | 55.182 | −3.058 | 1.00 | 57.49 |
| ATOM | 2486 | CZ3 | TRP | 50 | 43.267 | 54.872 | −4.132 | 1.00 | 57.49 |
| ATOM | 2487 | CH2 | TRP | 50 | 42.033 | 55.486 | −4.056 | 1.00 | 57.49 |
| ATOM | 2488 | C | TRP | 50 | 44.923 | 50.556 | −2.296 | 1.00 | 2.00 |
| ATOM | 2489 | O | TRP | 50 | 46.141 | 50.436 | −2.292 | 1.00 | 57.49 |
| ATOM | 2490 | N | ILE | 51 | 44.239 | 50.756 | −3.407 | 1.00 | 2.58 |
| ATOM | 2492 | CA | ILE | 51 | 44.957 | 50.921 | −4.652 | 1.00 | 2.58 |
| ATOM | 2493 | CB | ILE | 51 | 45.528 | 49.623 | −5.217 | 1.00 | 4.23 |
| ATOM | 2494 | CG2 | ILE | 51 | 44.516 | 48.983 | −6.161 | 1.00 | 4.23 |
| ATOM | 2495 | CG1 | ILE | 51 | 46.800 | 49.968 | −5.991 | 1.00 | 4.23 |
| ATOM | 2496 | CD1 | ILE | 51 | 47.581 | 48.788 | −6.481 | 1.00 | 4.23 |
| ATOM | 2497 | C | ILE | 51 | 44.113 | 51.616 | −5.693 | 1.00 | 2.58 |
| ATOM | 2498 | O | ILE | 51 | 42.925 | 51.332 | −5.854 | 1.00 | 4.23 |
| ATOM | 2499 | N | ASN | 52 | 44.738 | 52.546 | −6.398 | 1.00 | 33.49 |
| ATOM | 2501 | CA | ASN | 52 | 44.042 | 53.268 | −7.441 | 1.00 | 33.49 |
| ATOM | 2502 | CB | ASN | 52 | 44.451 | 54.725 | −7.525 | 1.00 | 15.27 |
| ATOM | 2503 | CG | ASN | 52 | 43.618 | 55.455 | −8.514 | 1.00 | 15.27 |
| ATOM | 2504 | OD1 | ASN | 52 | 43.668 | 55.173 | −9.715 | 1.00 | 15.27 |
| ATOM | 2505 | ND2 | ASN | 52 | 42.740 | 56.301 | −8.015 | 1.00 | 15.27 |
| ATOM | 2508 | C | ASN | 52 | 44.369 | 52.571 | −8.732 | 1.00 | 33.49 |
| ATOM | 2509 | O | ASN | 52 | 45.373 | 52.841 | −9.404 | 1.00 | 15.27 |
| ATOM | 2510 | N | THR | 53 | 43.386 | 51.808 | −9.129 | 1.00 | 16.45 |
| ATOM | 2512 | CA | THR | 53 | 43.414 | 50.928 | −10.257 | 1.00 | 16.45 |
| ATOM | 2513 | CB | THR | 53 | 42.142 | 50.216 | −10.205 | 1.00 | 42.20 |
| ATOM | 2514 | OG1 | THR | 53 | 41.089 | 51.138 | −10.536 | 1.00 | 42.20 |
| ATOM | 2516 | CG2 | THR | 53 | 41.936 | 49.718 | −8.773 | 1.00 | 42.20 |
| ATOM | 2517 | C | THR | 53 | 43.536 | 51.480 | −11.656 | 1.00 | 16.45 |
| ATOM | 2518 | O | THR | 53 | 42.981 | 50.923 | −12.616 | 1.00 | 42.20 |
| ATOM | 2519 | N | ARG | 54 | 44.229 | 52.583 | −11.795 | 1.00 | 50.54 |
| ATOM | 2521 | CA | ARG | 54 | 44.366 | 53.184 | −13.107 | 1.00 | 50.54 |
| ATOM | 2522 | CB | ARG | 54 | 43.377 | 54.373 | −13.131 | 1.00 | 42.70 |
| ATOM | 2523 | CG | ARG | 54 | 43.078 | 54.966 | −14.495 | 1.00 | 42.70 |
| ATOM | 2524 | CD | ARG | 54 | 43.317 | 56.486 | −14.569 | 1.00 | 42.70 |
| ATOM | 2525 | NE | ARG | 54 | 42.980 | 56.929 | −15.921 | 1.00 | 42.70 |
| ATOM | 2527 | CZ | ARG | 54 | 43.854 | 57.134 | −16.902 | 1.00 | 42.70 |
| ATOM | 2528 | NH1 | ARG | 54 | 45.163 | 56.985 | −16.697 | 1.00 | 42.70 |
| ATOM | 2531 | NH2 | ARG | 54 | 43.407 | 57.341 | −18.139 | 1.00 | 42.70 |
| ATOM | 2534 | C | ARG | 54 | 45.798 | 53.722 | −13.122 | 1.00 | 50.54 |
| ATOM | 2535 | O | ARG | 54 | 46.453 | 53.897 | −14.161 | 1.00 | 42.70 |
| ATOM | 2536 | N | ASN | 55 | 46.349 | 53.636 | −11.933 | 1.00 | 22.51 |
| ATOM | 2538 | CA | ASN | 55 | 47.588 | 54.260 | −11.622 | 1.00 | 22.51 |
| ATOM | 2539 | CB | ASN | 55 | 47.182 | 55.219 | −10.536 | 1.00 | 62.29 |
| ATOM | 2540 | CG | ASN | 55 | 48.043 | 56.422 | −10.448 | 1.00 | 62.29 |
| ATOM | 2541 | OD1 | ASN | 55 | 48.996 | 56.618 | −11.205 | 1.00 | 62.29 |
| ATOM | 2542 | ND2 | ASN | 55 | 47.679 | 57.279 | −9.517 | 1.00 | 62.29 |
| ATOM | 2545 | C | ASN | 55 | 48.594 | 53.325 | −11.040 | 1.00 | 22.51 |
| ATOM | 2546 | O | ASN | 55 | 49.771 | 53.374 | −11.369 | 1.00 | 62.29 |
| ATOM | 2547 | N | GLY | 56 | 48.129 | 52.529 | −10.088 | 1.00 | 49.54 |
| ATOM | 2549 | CA | GLY | 56 | 49.031 | 51.639 | −9.397 | 1.00 | 49.54 |
| ATOM | 2550 | C | GLY | 56 | 49.476 | 52.347 | −8.124 | 1.00 | 49.54 |
| ATOM | 2551 | O | GLY | 56 | 50.042 | 51.719 | −7.214 | 1.00 | 47.80 |
| ATOM | 2552 | N | LYS | 57 | 49.244 | 53.661 | −8.044 | 1.00 | 54.37 |
| ATOM | 2554 | CA | LYS | 57 | 49.608 | 54.400 | −6.833 | 1.00 | 54.37 |
| ATOM | 2555 | CB | LYS | 57 | 49.354 | 55.911 | −6.963 | 1.00 | 38.06 |
| ATOM | 2556 | CG | LYS | 57 | 50.526 | 56.635 | −7.654 | 1.00 | 38.06 |
| ATOM | 2557 | CD | LYS | 57 | 50.180 | 58.024 | −8.266 | 1.00 | 38.06 |
| ATOM | 2558 | CE | LYS | 57 | 50.217 | 59.176 | −7.281 | 1.00 | 38.06 |
| ATOM | 2559 | NZ | LYS | 57 | 51.151 | 60.258 | −7.772 | 1.00 | 38.06 |
| ATOM | 2563 | C | LYS | 57 | 48.819 | 53.662 | −5.761 | 1.00 | 54.37 |
| ATOM | 2564 | O | LYS | 57 | 47.726 | 53.131 | −6.030 | 1.00 | 38.06 |
| ATOM | 2565 | N | SER | 58 | 49.419 | 53.582 | −4.581 | 1.00 | 54.98 |
| ATOM | 2567 | CA | SER | 58 | 48.887 | 52.742 | −3.525 | 1.00 | 54.98 |
| ATOM | 2568 | CB | SER | 58 | 49.664 | 51.452 | −3.702 | 1.00 | 58.93 |
| ATOM | 2569 | OG | SER | 58 | 51.012 | 51.786 | −4.083 | 1.00 | 58.93 |
| ATOM | 2571 | C | SER | 58 | 49.025 | 53.181 | −2.050 | 1.00 | 54.98 |
| ATOM | 2572 | O | SER | 58 | 50.106 | 53.608 | −1.630 | 1.00 | 58.93 |
| ATOM | 2573 | N | THR | 59 | 47.982 | 52.953 | −1.247 | 1.00 | 34.76 |
| ATOM | 2575 | CA | THR | 59 | 47.991 | 53.360 | 0.163 | 1.00 | 34.76 |
| ATOM | 2576 | CB | THR | 59 | 46.808 | 54.265 | 0.424 | 1.00 | 50.04 |
| ATOM | 2577 | OG1 | THR | 59 | 46.669 | 55.185 | −0.672 | 1.00 | 50.04 |
| ATOM | 2579 | CG2 | THR | 59 | 47.012 | 55.055 | 1.720 | 1.00 | 50.04 |
| ATOM | 2580 | C | THR | 59 | 47.812 | 52.183 | 1.085 | 1.00 | 34.76 |
| ATOM | 2581 | O | THR | 59 | 46.880 | 51.425 | 0.888 | 1.00 | 50.04 |
| ATOM | 2582 | N | TYR | 60 | 48.648 | 52.037 | 2.111 | 1.00 | 21.76 |
| ATOM | 2584 | CA | TYR | 60 | 48.543 | 50.877 | 3.040 | 1.00 | 21.76 |
| ATOM | 2585 | CB | TYR | 60 | 49.768 | 49.964 | 2.990 | 1.00 | 21.25 |
| ATOM | 2586 | CG | TYR | 60 | 50.373 | 49.642 | 1.661 | 1.00 | 21.25 |
| ATOM | 2587 | CD1 | TYR | 60 | 49.743 | 49.934 | 0.468 | 1.00 | 21.25 |
| ATOM | 2588 | CE1 | TYR | 60 | 50.336 | 49.623 | −0.751 | 1.00 | 21.25 |
| ATOM | 2589 | CD2 | TYR | 60 | 51.614 | 49.022 | 1.600 | 1.00 | 21.25 |
| ATOM | 2590 | CE2 | TYR | 60 | 52.191 | 48.699 | 0.407 | 1.00 | 21.25 |
| ATOM | 2591 | CZ | TYR | 60 | 51.557 | 49.000 | −0.763 | 1.00 | 21.25 |
| ATOM | 2592 | OH | TYR | 60 | 52.147 | 48.629 | −1.923 | 1.00 | 21.25 |
| ATOM | 2594 | C | TYR | 60 | 48.452 | 51.284 | 4.495 | 1.00 | 21.76 |
| ATOM | 2595 | O | TYR | 60 | 49.056 | 52.274 | 4.882 | 1.00 | 21.25 |
| ATOM | 2596 | N | VAL | 61 | 47.793 | 50.459 | 5.307 | 1.00 | 2.00 |
| ATOM | 2598 | CA | VAL | 61 | 47.636 | 50.717 | 6.748 | 1.00 | 2.00 |
| ATOM | 2599 | CB | VAL | 61 | 46.724 | 49.642 | 7.436 | 1.00 | 36.32 |
| ATOM | 2600 | CG1 | VAL | 61 | 47.388 | 49.056 | 8.727 | 1.00 | 36.32 |
| ATOM | 2601 | CG2 | VAL | 61 | 45.318 | 50.258 | 7.783 | 1.00 | 36.32 |
| ATOM | 2602 | C | VAL | 61 | 48.997 | 50.684 | 7.395 | 1.00 | 2.00 |
| ATOM | 2603 | O | VAL | 61 | 49.909 | 50.132 | 6.812 | 1.00 | 36.32 |
| ATOM | 2604 | N | ASP | 62 | 49.126 | 51.225 | 8.610 | 1.00 | 69.13 |
| ATOM | 2606 | CA | ASP | 62 | 50.439 | 51.226 | 9.291 | 1.00 | 69.13 |
| ATOM | 2607 | CB | ASP | 62 | 50.443 | 52.071 | 10.580 | 1.00 | 34.42 |
| ATOM | 2608 | CG | ASP | 62 | 50.989 | 53.499 | 10.376 | 1.00 | 34.42 |
| ATOM | 2609 | OD1 | ASP | 62 | 51.241 | 54.198 | 11.375 | 1.00 | 34.42 |
| ATOM | 2610 | OD2 | ASP | 62 | 51.149 | 53.950 | 9.218 | 1.00 | 34.42 |
| ATOM | 2611 | C | ASP | 62 | 51.020 | 49.843 | 9.620 | 1.00 | 69.13 |
| ATOM | 2612 | O | ASP | 62 | 52.212 | 49.614 | 9.403 | 1.00 | 34.42 |
| ATOM | 2613 | N | ASP | 63 | 50.219 | 48.932 | 10.176 | 1.00 | 37.05 |
| ATOM | 2615 | CA | ASP | 63 | 50.841 | 47.653 | 10.476 | 1.00 | 37.05 |
| ATOM | 2616 | CB | ASP | 63 | 50.404 | 47.047 | 11.818 | 1.00 | 31.00 |
| ATOM | 2617 | CG | ASP | 63 | 49.130 | 47.638 | 12.344 | 1.00 | 31.00 |
| ATOM | 2618 | OD1 | ASP | 63 | 49.206 | 48.353 | 13.380 | 1.00 | 31.00 |
| ATOM | 2619 | OD2 | ASP | 63 | 48.083 | 47.396 | 11.705 | 1.00 | 31.00 |
| ATOM | 2620 | C | ASP | 63 | 50.729 | 46.662 | 9.365 | 1.00 | 37.05 |
| ATOM | 2621 | O | ASP | 63 | 50.195 | 45.574 | 9.558 | 1.00 | 31.00 |
| ATOM | 2622 | N | PHE | 64 | 51.151 | 47.070 | 8.179 | 1.00 | 9.67 |
| ATOM | 2624 | CA | PHE | 64 | 51.163 | 46.178 | 7.041 | 1.00 | 9.67 |
| ATOM | 2625 | CB | PHE | 64 | 49.824 | 46.205 | 6.333 | 1.00 | 25.09 |
| ATOM | 2626 | CG | PHE | 64 | 48.767 | 45.403 | 7.020 | 1.00 | 25.09 |
| ATOM | 2627 | CD1 | PHE | 64 | 47.897 | 45.998 | 7.930 | 1.00 | 25.09 |
| ATOM | 2628 | CD2 | PHE | 64 | 48.641 | 44.050 | 6.761 | 1.00 | 25.09 |
| ATOM | 2629 | CE1 | PHE | 64 | 46.931 | 45.264 | 8.573 | 1.00 | 25.09 |
| ATOM | 2630 | CE2 | PHE | 64 | 47.666 | 43.294 | 7.403 | 1.00 | 25.09 |
| ATOM | 2631 | CZ | PHE | 64 | 46.805 | 43.902 | 8.312 | 1.00 | 25.09 |
| ATOM | 2632 | C | PHE | 64 | 52.293 | 46.600 | 6.112 | 1.00 | 9.67 |
| ATOM | 2633 | O | PHE | 64 | 52.075 | 46.831 | 4.923 | 1.00 | 25.09 |
| ATOM | 2634 | N | LYS | 65 | 53.521 | 46.632 | 6.649 | 1.00 | 35.36 |
| ATOM | 2636 | CA | LYS | 65 | 54.705 | 47.077 | 5.895 | 1.00 | 35.36 |
| ATOM | 2637 | CB | LYS | 65 | 55.323 | 48.312 | 6.556 | 1.00 | 32.71 |
| ATOM | 2638 | CG | LYS | 65 | 54.338 | 49.329 | 7.073 | 1.00 | 32.71 |
| ATOM | 2639 | CD | LYS | 65 | 53.444 | 49.804 | 5.960 | 1.00 | 32.71 |
| ATOM | 2640 | CE | LYS | 65 | 54.174 | 50.709 | 5.006 | 1.00 | 32.71 |
| ATOM | 2641 | NZ | LYS | 65 | 53.726 | 52.139 | 5.153 | 1.00 | 32.71 |
| ATOM | 2645 | C | LYS | 65 | 55.847 | 46.104 | 5.692 | 1.00 | 35.36 |
| ATOM | 2646 | O | LYS | 65 | 56.414 | 45.574 | 6.651 | 1.00 | 32.71 |
| ATOM | 2647 | N | GLY | 66 | 56.262 | 45.981 | 4.431 | 1.00 | 89.30 |
| ATOM | 2649 | CA | GLY | 66 | 57.401 | 45.142 | 4.072 | 1.00 | 89.30 |
| ATOM | 2650 | C | GLY | 66 | 57.055 | 44.153 | 2.973 | 1.00 | 89.30 |
| ATOM | 2651 | O | GLY | 66 | 57.389 | 44.293 | 1.781 | 1.00 | 46.24 |

TABLE 5
Three dimensional coordinates of
HC - CDR3 (HC: GLU99-TYR110) from BC2

| | | | | | x | y | z | Q | B |
|---|---|---|---|---|---|---|---|---|---|
| ATOM | 2965 | N | GLU | 99 | 35.523 | 49.858 | −3.257 | 1.00 | 25.76 |
| ATOM | 2967 | CA | GLU | 99 | 34.886 | 51.032 | −2.668 | 1.00 | 25.76 |
| ATOM | 2968 | CB | GLU | 99 | 35.928 | 51.777 | −1.791 | 1.00 | 63.63 |
| ATOM | 2969 | CG | GLU | 99 | 35.878 | 53.373 | −1.706 | 1.00 | 63.63 |
| ATOM | 2970 | CD | GLU | 99 | 37.307 | 54.052 | −1.532 | 1.00 | 63.63 |
| ATOM | 2971 | OE1 | GLU | 99 | 38.278 | 53.384 | −1.090 | 1.00 | 63.63 |
| ATOM | 2972 | OE2 | GLU | 99 | 37.460 | 55.269 | −1.828 | 1.00 | 63.63 |
| ATOM | 2973 | C | GLU | 99 | 34.507 | 51.829 | −3.943 | 1.00 | 25.76 |
| ATOM | 2974 | O | GLU | 99 | 35.321 | 51.930 | −4.866 | 1.00 | 63.63 |
| ATOM | 2975 | N | GLY | 100 | 33.234 | 52.203 | −4.085 | 1.00 | 36.41 |
| ATOM | 2977 | CA | GLY | 100 | 32.814 | 52.965 | −5.257 | 1.00 | 36.41 |
| ATOM | 2978 | C | GLY | 100 | 31.914 | 54.127 | −4.831 | 1.00 | 36.41 |
| ATOM | 2979 | O | GLY | 100 | 31.060 | 53.923 | −3.995 | 1.00 | 41.46 |
| ATOM | 2980 | N | ASN | 101 | 31.966 | 55.277 | −5.502 | 1.00 | 33.66 |
| ATOM | 2982 | CA | ASN | 101 | 31.196 | 56.434 | −5.060 | 1.00 | 33.66 |
| ATOM | 2983 | CB | ASN | 101 | 31.810 | 57.744 | −5.534 | 1.00 | 24.12 |
| ATOM | 2984 | CG | ASN | 101 | 32.059 | 58.676 | −4.388 | 1.00 | 24.12 |
| ATOM | 2985 | OD1 | ASN | 101 | 31.122 | 59.065 | −3.700 | 1.00 | 24.12 |
| ATOM | 2986 | ND2 | ASN | 101 | 33.320 | 58.941 | −4.099 | 1.00 | 24.12 |
| ATOM | 2989 | C | ASN | 101 | 29.689 | 56.506 | −5.183 | 1.00 | 33.66 |
| ATOM | 2990 | O | ASN | 101 | 29.117 | 56.182 | −6.233 | 1.00 | 24.12 |
| ATOM | 2991 | N | MET | 102 | 29.083 | 57.024 | −4.102 | 1.00 | 83.69 |
| ATOM | 2993 | CA | MET | 102 | 27.625 | 57.284 | −3.908 | 1.00 | 83.69 |
| ATOM | 2994 | CB | MET | 102 | 26.730 | 56.030 | −4.143 | 1.00 | 59.11 |
| ATOM | 2995 | CG | MET | 102 | 25.270 | 56.204 | −3.635 | 1.00 | 59.11 |
| ATOM | 2996 | SD | MET | 102 | 23.981 | 55.029 | −4.261 | 1.00 | 59.11 |
| ATOM | 2997 | CE | MET | 102 | 22.477 | 56.146 | −4.344 | 1.00 | 59.11 |
| ATOM | 2998 | C | MET | 102 | 27.430 | 57.829 | −2.459 | 1.00 | 83.69 |
| ATOM | 2999 | O | MET | 102 | 27.367 | 57.011 | −1.513 | 1.00 | 59.11 |
| ATOM | 3000 | N | ASP | 103 | 27.313 | 59.177 | −2.335 | 1.00 | 81.57 |
| ATOM | 3002 | CA | ASP | 103 | 27.125 | 59.990 | −1.086 | 1.00 | 81.57 |
| ATOM | 3003 | CB | ASP | 103 | 26.625 | 59.139 | 0.117 | 1.00 | 22.70 |
| ATOM | 3004 | CG | ASP | 103 | 26.176 | 59.987 | 1.343 | 1.00 | 22.70 |
| ATOM | 3005 | OD1 | ASP | 103 | 26.907 | 60.892 | 1.813 | 1.00 | 22.70 |
| ATOM | 3006 | OD2 | ASP | 103 | 25.106 | 59.666 | 1.902 | 1.00 | 22.70 |
| ATOM | 3007 | C | ASP | 103 | 28.446 | 60.681 | −0.759 | 1.00 | 81.57 |
| ATOM | 3008 | O | ASP | 103 | 28.961 | 60.589 | 0.366 | 1.00 | 22.70 |
| ATOM | 3009 | N | GLY | 104 | 28.984 | 61.379 | −1.761 | 1.00 | 86.53 |
| ATOM | 3011 | CA | GLY | 104 | 30.272 | 62.065 | −1.608 | 1.00 | 86.53 |
| ATOM | 3012 | C | GLY | 104 | 31.473 | 61.122 | −1.460 | 1.00 | 86.53 |
| ATOM | 3013 | O | GLY | 104 | 32.234 | 60.889 | −2.412 | 1.00 | 46.20 |
| ATOM | 3014 | N | TYR | 105 | 31.716 | 60.669 | −0.228 | 1.00 | 98.58 |
| ATOM | 3016 | CA | TYR | 105 | 32.808 | 59.688 | 0.036 | 1.00 | 98.58 |
| ATOM | 3017 | CB | TYR | 105 | 33.017 | 59.412 | 1.557 | 1.00 | 64.10 |
| ATOM | 3018 | CG | TYR | 105 | 33.326 | 60.523 | 2.585 | 1.00 | 64.10 |
| ATOM | 3019 | CD1 | TYR | 105 | 34.643 | 60.884 | 2.881 | 1.00 | 64.10 |
| ATOM | 3020 | CE1 | TYR | 105 | 34.952 | 61.681 | 3.977 | 1.00 | 64.10 |
| ATOM | 3021 | CD2 | TYR | 105 | 32.318 | 61.015 | 3.435 | 1.00 | 64.10 |
| ATOM | 3022 | CE2 | TYR | 105 | 32.620 | 61.810 | 4.531 | 1.00 | 64.10 |
| ATOM | 3023 | CZ | TYR | 105 | 33.936 | 62.128 | 4.802 | 1.00 | 64.10 |
| ATOM | 3024 | OH | TYR | 105 | 34.269 | 62.843 | 5.920 | 1.00 | 64.10 |
| ATOM | 3026 | C | TYR | 105 | 32.256 | 58.342 | −0.529 | 1.00 | 93.58 |
| ATOM | 3027 | O | TYR | 105 | 31.153 | 58.311 | −1.096 | 1.00 | 64.10 |
| ATOM | 3028 | N | PHE | 106 | 32.944 | 57.243 | −0.170 | 1.00 | 48.28 |
| ATOM | 3030 | CA | PHE | 106 | 32.570 | 55.829 | −0.484 | 1.00 | 48.28 |
| ATOM | 3031 | CB | PHE | 106 | 32.058 | 55.183 | 0.800 | 1.00 | 53.07 |
| ATOM | 3032 | CG | PHE | 106 | 30.689 | 55.652 | 1.196 | 1.00 | 53.07 |
| ATOM | 3033 | CD1 | PHE | 106 | 30.486 | 56.963 | 1.576 | 1.00 | 53.07 |
| ATOM | 3034 | CD2 | PHE | 106 | 29.597 | 54.794 | 1.117 | 1.00 | 53.07 |
| ATOM | 3035 | CE1 | PHE | 106 | 29.255 | 57.405 | 1.875 | 1.00 | 53.07 |
| ATOM | 3036 | CE2 | PHE | 106 | 28.347 | 55.232 | 1.414 | 1.00 | 53.07 |
| ATOM | 3037 | CZ | PHE | 106 | 28.161 | 56.548 | 1.791 | 1.00 | 53.07 |
| ATOM | 3038 | C | PHE | 106 | 31.498 | 55.596 | −1.605 | 1.00 | 48.28 |
| ATOM | 3039 | O | PHE | 106 | 31.316 | 56.481 | −2.420 | 1.00 | 53.07 |
| ATOM | 3040 | N | PRO | 107 | 30.807 | 54.401 | −1.651 | 1.00 | 76.34 |
| ATOM | 3041 | CD | PRO | 107 | 29.472 | 54.859 | −2.148 | 1.00 | 42.84 |
| ATOM | 3042 | CA | PRO | 107 | 30.633 | 53.080 | −0.965 | 1.00 | 76.34 |
| ATOM | 3043 | CB | PRO | 107 | 29.321 | 52.561 | −1.567 | 1.00 | 42.84 |
| ATOM | 3044 | CG | PRO | 107 | 28.479 | 53.811 | −1.600 | 1.00 | 42.84 |
| ATOM | 3045 | C | PRO | 107 | 31.730 | 51.933 | −0.822 | 1.00 | 76.34 |
| ATOM | 3046 | O | PRO | 107 | 32.951 | 52.163 | −0.993 | 1.00 | 42.84 |
| ATOM | 3047 | N | PHE | 108 | 31.227 | 50.700 | −0.638 | 1.00 | 52.21 |
| ATOM | 3049 | CA | PHE | 108 | 31.951 | 49.437 | −0.323 | 1.00 | 52.21 |
| ATOM | 3050 | CB | PHE | 108 | 31.919 | 49.332 | 1.174 | 1.00 | 28.72 |
| ATOM | 3051 | CG | PHE | 108 | 30.743 | 50.065 | 1.736 | 1.00 | 28.72 |
| ATOM | 3052 | CD1 | PHE | 108 | 30.900 | 51.325 | 2.267 | 1.00 | 28.72 |
| ATOM | 3053 | CD2 | PHE | 108 | 29.464 | 49.611 | 1.445 | 1.00 | 28.72 |
| ATOM | 3054 | CE1 | PHE | 108 | 29.788 | 52.112 | 2.467 | 1.00 | 28.72 |
| ATOM | 3055 | CE2 | PHE | 108 | 28.351 | 50.384 | 1.636 | 1.00 | 28.72 |
| ATOM | 3056 | CZ | PHE | 108 | 28.508 | 51.635 | 2.135 | 1.00 | 28.72 |
| ATOM | 3057 | C | PHE | 108 | 30.973 | 48.375 | −0.826 | 1.00 | 52.21 |
| ATOM | 3058 | O | PHE | 108 | 30.487 | 47.516 | −0.077 | 1.00 | 28.72 |
| ATOM | 3059 | N | THR | 109 | 30.699 | 48.439 | −2.115 | 1.00 | 26.26 |
| ATOM | 3061 | CA | THR | 109 | 29.735 | 47.613 | −2.797 | 1.00 | 26.26 |
| ATOM | 3062 | CB | THR | 109 | 29.620 | 48.129 | −4.186 | 1.00 | 36.21 |
| ATOM | 3063 | OG1 | THR | 109 | 30.948 | 48.431 | −4.661 | 1.00 | 36.21 |
| ATOM | 3065 | CG2 | THR | 109 | 28.723 | 49.376 | −4.229 | 1.00 | 36.21 |
| ATOM | 3066 | C | THR | 109 | 29.831 | 46.122 | −2.998 | 1.00 | 26.26 |
| ATOM | 3067 | O | THR | 109 | 28.942 | 45.377 | −2.617 | 1.00 | 36.21 |
| ATOM | 3068 | N | TYR | 110 | 30.817 | 45.735 | −3.796 | 1.00 | 20.44 |
| ATOM | 3070 | CA | TYR | 110 | 31.000 | 44.328 | −4.171 | 1.00 | 20.44 |
| ATOM | 3071 | CB | TYR | 110 | 30.912 | 44.207 | −5.686 | 1.00 | 60.15 |
| ATOM | 3072 | CG | TYR | 110 | 29.897 | 45.158 | −6.284 | 1.00 | 60.15 |
| ATOM | 3073 | CD1 | TYR | 110 | 28.578 | 45.154 | −5.841 | 1.00 | 60.15 |
| ATOM | 3074 | CE1 | TYR | 110 | 27.628 | 45.978 | −6.424 | 1.00 | 60.15 |
| ATOM | 3075 | CD2 | TYR | 110 | 30.246 | 46.025 | −7.321 | 1.00 | 60.15 |
| ATOM | 3076 | CE2 | TYR | 110 | 29.315 | 46.848 | −7.903 | 1.00 | 60.15 |
| ATOM | 3077 | CZ | TYR | 110 | 27.998 | 46.822 | −7.470 | 1.00 | 60.15 |
| ATOM | 3078 | OH | TYR | 110 | 27.074 | 47.577 | −8.158 | 1.00 | 60.15 |
| ATOM | 3080 | C | TYR | 110 | 32.284 | 43.665 | −3.691 | 1.00 | 20.44 |
| ATOM | 3081 | O | TYR | 110 | 33.283 | 43.680 | −4.404 | 1.00 | 60.15 |

TABLE 6
Three dimensional coordinates of
LC - CDR1 (LC: ARG24-HIS33) from BC2

| | | | | | x | y | z | Q | B |
|---|---|---|---|---|---|---|---|---|---|
| ATOM | 199 | N | ARG | 24 | 31.034 | 53.669 | 19.975 | 1.00 | 35.70 |
| ATOM | 201 | CA | ARG | 24 | 31.810 | 54.840 | 20.383 | 1.00 | 35.70 |
| ATOM | 202 | CB | ARG | 24 | 32.226 | 54.801 | 21.876 | 1.00 | 43.83 |
| ATOM | 203 | CG | ARG | 24 | 31.253 | 54.267 | 22.939 | 1.00 | 43.83 |
| ATOM | 204 | CD | ARG | 24 | 31.676 | 54.727 | 24.383 | 1.00 | 43.83 |
| ATOM | 205 | NE | ARG | 24 | 33.056 | 54.377 | 24.755 | 1.00 | 43.83 |
| ATOM | 207 | CZ | ARG | 24 | 33.426 | 53.850 | 25.931 | 1.00 | 43.83 |
| ATOM | 208 | NH1 | ARG | 24 | 32.531 | 53.605 | 26.891 | 1.00 | 43.83 |
| ATOM | 211 | NH2 | ARG | 24 | 34.697 | 53.526 | 26.132 | 1.00 | 43.83 |
| ATOM | 214 | C | ARG | 24 | 33.123 | 54.991 | 19.621 | 1.00 | 35.70 |
| ATOM | 215 | O | ARG | 24 | 33.959 | 54.092 | 19.630 | 1.00 | 43.83 |
| ATOM | 216 | N | ALA | 25 | 33.326 | 56.123 | 18.974 | 1.00 | 82.87 |
| ATOM | 218 | CA | ALA | 25 | 34.622 | 56.346 | 18.320 | 1.00 | 82.87 |
| ATOM | 219 | CB | ALA | 25 | 34.436 | 57.225 | 17.056 | 1.00 | 87.02 |
| ATOM | 220 | C | ALA | 25 | 35.461 | 57.105 | 19.369 | 1.00 | 82.87 |
| ATOM | 221 | O | ALA | 25 | 34.882 | 57.853 | 20.152 | 1.00 | 87.02 |
| ATOM | 222 | N | SER | 26 | 36.786 | 56.920 | 19.422 | 1.00 | 44.67 |
| ATOM | 224 | CA | SER | 26 | 37.565 | 57.688 | 20.410 | 1.00 | 44.67 |
| ATOM | 225 | CB | SER | 26 | 39.000 | 57.177 | 20.557 | 1.00 | 4.82 |
| ATOM | 226 | OG | SER | 26 | 39.698 | 57.261 | 19.336 | 1.00 | 4.82 |
| ATOM | 228 | C | SER | 26 | 37.582 | 59.186 | 20.040 | 1.00 | 44.67 |
| ATOM | 229 | O | SER | 26 | 37.708 | 60.047 | 20.912 | 1.00 | 4.82 |
| ATOM | 230 | N | SER | 27 | 37.430 | 59.501 | 18.755 | 1.00 | 27.16 |
| ATOM | 232 | CA | SER | 27 | 37.462 | 60.916 | 18.351 | 1.00 | 27.16 |
| ATOM | 233 | CB | SER | 27 | 38.837 | 61.282 | 17.765 | 1.00 | 37.32 |
| ATOM | 234 | OG | SER | 27 | 39.886 | 61.091 | 18.724 | 1.00 | 37.32 |
| ATOM | 236 | C | SER | 27 | 36.374 | 61.225 | 17.362 | 1.00 | 27.16 |
| ATOM | 237 | O | SER | 27 | 35.718 | 60.310 | 16.860 | 1.00 | 37.32 |
| ATOM | 238 | N | SER | 28 | 36.185 | 62.501 | 17.060 | 1.00 | 32.79 |
| ATOM | 240 | CA | SER | 28 | 35.117 | 62.876 | 16.134 | 1.00 | 32.79 |
| ATOM | 241 | CB | SER | 28 | 34.817 | 64.378 | 16.238 | 1.00 | 44.89 |
| ATOM | 242 | OG | SER | 28 | 34.248 | 64.686 | 17.509 | 1.00 | 44.89 |
| ATOM | 244 | C | SER | 28 | 35.316 | 62.487 | 14.671 | 1.00 | 32.79 |
| ATOM | 245 | O | SER | 28 | 36.334 | 62.847 | 14.060 | 1.00 | 44.89 |
| ATOM | 246 | N | VAL | 29 | 34.333 | 61.749 | 14.132 | 1.00 | 25.47 |
| ATOM | 248 | CA | VAL | 29 | 34.322 | 61.309 | 12.731 | 1.00 | 25.47 |
| ATOM | 249 | CB | VAL | 29 | 34.592 | 59.832 | 12.597 | 1.00 | 6.08 |
| ATOM | 250 | CG1 | VAL | 29 | 33.479 | 59.053 | 13.249 | 1.00 | 6.08 |
| ATOM | 251 | CG2 | VAL | 29 | 34.735 | 59.486 | 11.152 | 1.00 | 6.08 |
| ATOM | 252 | C | VAL | 29 | 32.990 | 61.664 | 12.049 | 1.00 | 25.47 |
| ATOM | 253 | O | VAL | 29 | 31.974 | 61.820 | 12.715 | 1.00 | 6.08 |
| ATOM | 254 | N | ASN | 30 | 32.994 | 61.694 | 10.711 | 1.00 | 14.73 |
| ATOM | 256 | CA | ASN | 30 | 31.843 | 62.139 | 9.908 | 1.00 | 14.73 |
| ATOM | 257 | CB | ASN | 30 | 32.372 | 62.765 | 8.606 | 1.00 | 54.87 |
| ATOM | 258 | CG | ASN | 30 | 33.253 | 64.006 | 8.853 | 1.00 | 54.87 |
| ATOM | 259 | OD1 | ASN | 30 | 33.627 | 64.730 | 7.915 | 1.00 | 54.87 |
| ATOM | 260 | ND2 | ASN | 30 | 33.581 | 64.265 | 10.123 | 1.00 | 54.87 |
| ATOM | 263 | C | ASN | 30 | 30.530 | 61.380 | 9.587 | 1.00 | 14.73 |
| ATOM | 264 | O | ASN | 30 | 29.515 | 62.046 | 9.304 | 1.00 | 54.87 |
| ATOM | 265 | N | TYR | 31 | 30.508 | 60.040 | 9.619 | 1.00 | 32.69 |
| ATOM | 267 | CA | TYR | 31 | 29.296 | 59.231 | 9.272 | 1.00 | 32.69 |
| ATOM | 268 | CB | TYR | 31 | 28.842 | 59.474 | 7.827 | 1.00 | 35.47 |
| ATOM | 269 | CG | TYR | 31 | 29.807 | 58.968 | 6.782 | 1.00 | 35.47 |
| ATOM | 270 | CD1 | TYR | 31 | 29.369 | 58.639 | 5.509 | 1.00 | 35.47 |
| ATOM | 271 | CE1 | TYR | 31 | 30.253 | 58.276 | 4.526 | 1.00 | 35.47 |
| ATOM | 272 | CD2 | TYR | 31 | 31.180 | 58.883 | 7.021 | 1.00 | 35.47 |
| ATOM | 273 | CE2 | TYR | 31 | 32.065 | 58.497 | 6.034 | 1.00 | 35.47 |
| ATOM | 274 | CZ | TYR | 31 | 31.597 | 58.200 | 4.776 | 1.00 | 35.47 |
| ATOM | 275 | OH | TYR | 31 | 32.441 | 57.819 | 3.774 | 1.00 | 35.47 |
| ATOM | 277 | C | TYR | 31 | 29.598 | 57.764 | 9.380 | 1.00 | 32.69 |
| ATOM | 278 | O | TYR | 31 | 30.758 | 57.393 | 9.362 | 1.00 | 35.47 |
| ATOM | 279 | N | MET | 32 | 28.582 | 56.902 | 9.311 | 1.00 | 32.43 |
| ATOM | 281 | CA | MET | 32 | 28.871 | 55.457 | 9.421 | 1.00 | 32.43 |
| ATOM | 282 | CB | MET | 32 | 28.762 | 54.944 | 10.841 | 1.00 | 25.19 |
| ATOM | 283 | CG | MET | 32 | 30.091 | 54.566 | 11.416 | 1.00 | 25.19 |
| ATOM | 284 | SD | MET | 32 | 29.802 | 53.661 | 12.911 | 1.00 | 25.19 |
| ATOM | 285 | CE | MET | 32 | 30.987 | 54.323 | 14.048 | 1.00 | 25.19 |
| ATOM | 286 | C | MET | 32 | 28.286 | 54.415 | 8.494 | 1.00 | 32.43 |
| ATOM | 287 | O | MET | 32 | 27.156 | 54.486 | 8.031 | 1.00 | 25.19 |
| ATOM | 288 | N | HIS | 33 | 29.094 | 53.397 | 8.266 | 1.00 | 41.58 |
| ATOM | 290 | CA | HIS | 33 | 28.729 | 52.285 | 7.411 | 1.00 | 41.58 |
| ATOM | 291 | CB | HIS | 33 | 29.763 | 52.135 | 6.303 | 1.00 | 27.09 |
| ATOM | 292 | CG | HIS | 33 | 29.889 | 53.329 | 5.438 | 1.00 | 27.09 |
| ATOM | 293 | CD2 | HIS | 33 | 28.963 | 54.054 | 4.784 | 1.00 | 27.09 |
| ATOM | 294 | ND1 | HIS | 33 | 31.084 | 53.947 | 5.213 | 1.00 | 27.09 |
| ATOM | 296 | CE1 | HIS | 33 | 30.912 | 55.005 | 4.445 | 1.00 | 27.09 |
| ATOM | 297 | NE2 | HIS | 33 | 29.619 | 55.085 | 4.178 | 1.00 | 27.09 |
| ATOM | 299 | C | HIS | 33 | 28.741 | 51.040 | 8.265 | 1.00 | 41.58 |
| ATOM | 300 | O | HIS | 33 | 29.751 | 50.763 | 8.934 | 1.00 | 27.09 |

TABLE 7
Three dimensional coordinates of
LC - CDR2 (ALA49-SER55) from BC2

| | | | | | x | y | z | Q | B |
|---|---|---|---|---|---|---|---|---|---|
| ATOM | 462 | N | ALA | 49 | 26.073 | 55.473 | 5.034 | 1.00 | 33.29 |
| ATOM | 464 | CA | ALA | 49 | 25.852 | 56.839 | 5.537 | 1.00 | 33.29 |
| ATOM | 465 | CB | ALA | 49 | 25.280 | 57.702 | 4.416 | 1.00 | 20.38 |
| ATOM | 466 | C | ALA | 49 | 24.957 | 56.935 | 6.776 | 1.00 | 33.29 |
| ATOM | 467 | O | ALA | 49 | 23.917 | 57.578 | 6.722 | 1.00 | 20.38 |
| ATOM | 468 | N | THR | 50 | 25.356 | 56.269 | 7.854 | 1.00 | 27.27 |
| ATOM | 470 | CA | THR | 50 | 24.647 | 56.254 | 9.128 | 1.00 | 27.27 |
| ATOM | 471 | CB | THR | 50 | 24.727 | 57.596 | 9.824 | 1.00 | 38.91 |
| ATOM | 472 | OG1 | THR | 50 | 26.101 | 57.970 | 9.914 | 1.00 | 38.91 |
| ATOM | 474 | CG2 | THR | 50 | 24.118 | 57.496 | 11.237 | 1.00 | 38.91 |
| ATOM | 475 | C | THR | 50 | 23.205 | 55.813 | 9.182 | 1.00 | 27.27 |
| ATOM | 476 | O | THR | 50 | 22.882 | 54.921 | 9.943 | 1.00 | 38.91 |
| ATOM | 477 | N | SER | 51 | 22.320 | 56.513 | 8.481 | 1.00 | 17.32 |
| ATOM | 479 | CA | SER | 51 | 20.912 | 56.148 | 8.500 | 1.00 | 17.32 |
| ATOM | 480 | CB | SER | 51 | 20.080 | 57.295 | 9.084 | 1.00 | 61.11 |
| ATOM | 481 | OG | SER | 51 | 20.699 | 57.780 | 10.281 | 1.00 | 61.11 |
| ATOM | 483 | C | SER | 51 | 20.422 | 55.717 | 7.121 | 1.00 | 17.32 |
| ATOM | 484 | O | SER | 51 | 19.258 | 55.405 | 6.945 | 1.00 | 61.11 |
| ATOM | 485 | N | ASN | 52 | 21.304 | 55.713 | 6.139 | 1.00 | 28.10 |
| ATOM | 487 | CA | ASN | 52 | 20.921 | 55.255 | 4.809 | 1.00 | 28.10 |
| ATOM | 488 | CB | ASN | 52 | 21.851 | 55.873 | 3.788 | 1.00 | 33.84 |
| ATOM | 489 | CG | ASN | 52 | 21.631 | 57.348 | 3.607 | 1.00 | 33.84 |
| ATOM | 490 | OD1 | ASN | 52 | 20.881 | 58.004 | 4.349 | 1.00 | 33.84 |
| ATOM | 491 | ND2 | ASN | 52 | 22.323 | 57.893 | 2.620 | 1.00 | 33.84 |
| ATOM | 494 | C | ASN | 52 | 20.954 | 53.713 | 4.650 | 1.00 | 28.10 |
| ATOM | 495 | O | ASN | 52 | 22.032 | 53.113 | 4.686 | 1.00 | 33.84 |
| ATOM | 496 | N | LEU | 53 | 19.797 | 53.084 | 4.392 | 1.00 | 46.86 |
| ATOM | 498 | CA | LEU | 53 | 19.714 | 51.607 | 4.228 | 1.00 | 46.86 |
| ATOM | 499 | CB | LEU | 53 | 18.296 | 51.079 | 4.477 | 1.00 | 14.22 |
| ATOM | 500 | CG | LEU | 53 | 17.803 | 51.184 | 5.911 | 1.00 | 14.22 |
| ATOM | 501 | CD1 | LEU | 53 | 16.468 | 50.481 | 6.075 | 1.00 | 14.22 |
| ATOM | 502 | CD2 | LEU | 53 | 18.826 | 50.577 | 6.823 | 1.00 | 14.22 |
| ATOM | 503 | C | LEU | 53 | 20.224 | 51.072 | 2.880 | 1.00 | 46.86 |
| ATOM | 504 | O | LEU | 53 | 20.184 | 51.769 | 1.857 | 1.00 | 14.22 |
| ATOM | 505 | N | ALA | 54 | 20.731 | 49.838 | 2.911 | 1.00 | 41.00 |
| ATOM | 507 | CA | ALA | 54 | 21.272 | 49.153 | 1.737 | 1.00 | 41.00 |
| ATOM | 508 | CB | ALA | 54 | 22.309 | 48.157 | 2.174 | 1.00 | 26.54 |
| ATOM | 509 | C | ALA | 54 | 20.166 | 48.465 | 0.946 | 1.00 | 41.00 |
| ATOM | 510 | O | ALA | 54 | 19.073 | 48.220 | 1.460 | 1.00 | 26.54 |
| ATOM | 511 | N | SER | 55 | 20.480 | 48.052 | −0.272 | 1.00 | 19.96 |
| ATOM | 513 | CA | SER | 55 | 19.452 | 47.470 | −1.097 | 1.00 | 19.96 |
| ATOM | 514 | CB | SER | 55 | 19.787 | 47.612 | −2.576 | 1.00 | 64.54 |
| ATOM | 515 | OG | SER | 55 | 18.587 | 47.553 | −3.340 | 1.00 | 64.54 |
| ATOM | 517 | C | SER | 55 | 19.037 | 46.060 | −0.792 | 1.00 | 19.96 |
| ATOM | 518 | O | SER | 55 | 19.652 | 45.088 | −1.257 | 1.00 | 64.54 |

TABLE 8
Three dimensional coordinates of LC - CDR3 (GLN88-THR96) from
BC2

| | | | | | x | y | z | Q | B |
|---|---|---|---|---|---|---|---|---|---|
| ATOM | 803 | N | GLN | 88 | 31.968 | 50.434 | 10.331 | 1.00 | 11.01 |
| ATOM | 805 | CA | GLN | 88 | 33.222 | 50.903 | 9.776 | 1.00 | 11.01 |
| ATOM | 806 | CB | GLN | 88 | 33.420 | 50.334 | 8.398 | 1.00 | 23.74 |
| ATOM | 807 | CG | GLN | 88 | 34.485 | 50.965 | 7.564 | 1.00 | 23.74 |
| ATOM | 808 | CD | GLN | 88 | 33.951 | 51.156 | 6.176 | 1.00 | 23.74 |
| ATOM | 809 | OE1 | GLN | 88 | 32.768 | 51.520 | 6.006 | 1.00 | 23.74 |
| ATOM | 810 | NE2 | GLN | 88 | 34.780 | 50.887 | 5.164 | 1.00 | 23.74 |
| ATOM | 813 | C | GLN | 88 | 33.131 | 52.420 | 9.743 | 1.00 | 11.01 |
| ATOM | 814 | O | GLN | 88 | 32.034 | 52.987 | 9.802 | 1.00 | 23.74 |
| ATOM | 815 | N | GLN | 89 | 34.289 | 53.063 | 9.641 | 1.00 | 22.56 |
| ATOM | 817 | CA | GLN | 89 | 34.453 | 54.515 | 9.651 | 1.00 | 22.56 |
| ATOM | 818 | CB | GLN | 89 | 35.447 | 54.806 | 10.813 | 1.00 | 21.80 |
| ATOM | 819 | CG | GLN | 89 | 36.354 | 56.035 | 10.763 | 1.00 | 21.80 |
| ATOM | 820 | CD | GLN | 89 | 37.702 | 55.805 | 10.084 | 1.00 | 21.80 |
| ATOM | 821 | OE1 | GLN | 89 | 37.886 | 56.146 | 8.907 | 1.00 | 21.80 |
| ATOM | 822 | NE2 | GLN | 89 | 38.650 | 55.247 | 10.817 | 1.00 | 21.80 |
| ATOM | 825 | C | GLN | 89 | 34.989 | 54.900 | 8.266 | 1.00 | 22.56 |
| ATOM | 826 | O | GLN | 89 | 35.529 | 54.045 | 7.606 | 1.00 | 21.80 |
| ATOM | 827 | N | TRP | 90 | 34.781 | 56.120 | 7.772 | 1.00 | 27.74 |
| ATOM | 829 | CA | TRP | 90 | 35.345 | 56.493 | 6.449 | 1.00 | 27.74 |
| ATOM | 830 | CB | TRP | 90 | 34.369 | 56.131 | 5.308 | 1.00 | 90.21 |
| ATOM | 831 | CG | TRP | 90 | 34.940 | 55.660 | 3.942 | 1.00 | 90.21 |
| ATOM | 832 | CD2 | TRP | 90 | 35.677 | 56.438 | 3.003 | 1.00 | 90.21 |
| ATOM | 833 | CE2 | TRP | 90 | 35.840 | 55.671 | 1.829 | 1.00 | 90.21 |
| ATOM | 834 | CE3 | TRP | 90 | 36.214 | 57.722 | 3.022 | 1.00 | 90.21 |
| ATOM | 835 | CD1 | TRP | 90 | 34.714 | 54.453 | 3.320 | 1.00 | 90.21 |
| ATOM | 836 | NE1 | TRP | 90 | 35.249 | 54.456 | 2.041 | 1.00 | 90.21 |
| ATOM | 838 | CZ2 | TRP | 90 | 36.510 | 56.156 | 0.702 | 1.00 | 90.21 |
| ATOM | 839 | CZ3 | TRP | 90 | 36.884 | 58.194 | 1.890 | 1.00 | 90.21 |
| ATOM | 840 | CH2 | TRP | 90 | 37.019 | 57.413 | 0.752 | 1.00 | 90.21 |
| ATOM | 841 | C | TRP | 90 | 35.614 | 57.999 | 6.437 | 1.00 | 27.74 |
| ATOM | 842 | O | TRP | 90 | 34.962 | 58.721 | 5.694 | 1.00 | 90.21 |
| ATOM | 843 | N | SER | 91 | 36.590 | 58.456 | 7.236 | 1.00 | 33.90 |
| ATOM | 845 | CA | SER | 91 | 36.919 | 59.882 | 7.305 | 1.00 | 33.90 |
| ATOM | 846 | CB | SER | 91 | 35.972 | 60.566 | 8.290 | 1.00 | 33.23 |
| ATOM | 847 | OG | SER | 91 | 34.617 | 60.159 | 8.093 | 1.00 | 33.23 |
| ATOM | 849 | C | SER | 91 | 38.345 | 60.167 | 7.787 | 1.00 | 33.90 |
| ATOM | 850 | O | SER | 91 | 38.725 | 61.333 | 7.955 | 1.00 | 33.23 |
| ATOM | 851 | N | ILE | 92 | 39.144 | 59.128 | 7.999 | 1.00 | 2.00 |
| ATOM | 853 | CA | ILE | 92 | 40.460 | 59.355 | 8.562 | 1.00 | 2.00 |
| ATOM | 854 | CB | ILE | 92 | 40.486 | 58.910 | 10.044 | 1.00 | 6.47 |
| ATOM | 855 | CG2 | ILE | 92 | 41.380 | 59.809 | 10.888 | 1.00 | 6.47 |
| ATOM | 856 | CG1 | ILE | 92 | 39.063 | 58.849 | 10.607 | 1.00 | 6.47 |
| ATOM | 857 | CD1 | ILE | 92 | 38.423 | 60.168 | 10.911 | 1.00 | 6.47 |
| ATOM | 858 | C | ILE | 92 | 41.495 | 58.514 | 7.947 | 1.00 | 2.00 |
| ATOM | 859 | O | ILE | 92 | 41.199 | 57.590 | 7.204 | 1.00 | 6.47 |
| ATOM | 860 | N | ASN | 93 | 42.732 | 58.864 | 8.266 | 1.00 | 50.27 |
| ATOM | 862 | CA | ASN | 93 | 43.854 | 58.038 | 7.897 | 1.00 | 50.27 |
| ATOM | 863 | CB | ASN | 93 | 45.208 | 58.800 | 7.682 | 1.00 | 86.79 |
| ATOM | 864 | CG | ASN | 93 | 46.486 | 57.828 | 7.455 | 1.00 | 86.79 |
| ATOM | 865 | OD1 | ASN | 93 | 47.427 | 57.772 | 8.308 | 1.00 | 86.79 |
| ATOM | 866 | ND2 | ASN | 93 | 46.515 | 57.093 | 6.321 | 1.00 | 86.79 |
| ATOM | 869 | C | ASN | 93 | 43.951 | 57.245 | 9.226 | 1.00 | 50.27 |
| ATOM | 870 | O | ASN | 93 | 43.982 | 57.844 | 10.306 | 1.00 | 86.79 |
| ATOM | 871 | N | PRO | 94 | 43.557 | 55.965 | 9.198 | 1.00 | 31.00 |
| ATOM | 872 | CD | PRO | 94 | 44.264 | 54.985 | 10.018 | 1.00 | 20.78 |
| ATOM | 873 | CA | PRO | 94 | 43.071 | 55.322 | 7.987 | 1.00 | 31.00 |
| ATOM | 874 | CB | PRO | 94 | 43.911 | 54.060 | 7.900 | 1.00 | 20.78 |
| ATOM | 875 | CG | PRO | 94 | 45.051 | 54.288 | 8.974 | 1.00 | 20.78 |
| ATOM | 876 | C | PRO | 94 | 41.636 | 55.034 | 8.421 | 1.00 | 31.00 |
| ATOM | 877 | O | PRO | 94 | 41.243 | 55.377 | 9.550 | 1.00 | 20.78 |
| ATOM | 878 | N | ARG | 95 | 40.833 | 54.492 | 7.530 | 1.00 | 12.98 |
| ATOM | 880 | CA | ARG | 95 | 39.478 | 54.164 | 7.925 | 1.00 | 12.98 |
| ATOM | 881 | CB | ARG | 95 | 38.592 | 54.022 | 6.711 | 1.00 | 25.66 |
| ATOM | 882 | CG | ARG | 95 | 39.316 | 53.550 | 5.504 | 1.00 | 25.66 |
| ATOM | 883 | CD | ARG | 95 | 38.629 | 54.020 | 4.254 | 1.00 | 25.66 |
| ATOM | 884 | NE | ARG | 95 | 39.628 | 54.435 | 3.283 | 1.00 | 25.66 |
| ATOM | 886 | CZ | ARG | 95 | 39.431 | 54.489 | 1.973 | 1.00 | 25.66 |
| ATOM | 887 | NH1 | ARG | 95 | 38.274 | 54.150 | 1.454 | 1.00 | 25.66 |
| ATOM | 890 | NH2 | ARG | 95 | 40.412 | 54.885 | 1.183 | 1.00 | 25.66 |
| ATOM | 893 | C | ARG | 95 | 39.599 | 52.868 | 8.709 | 1.00 | 12.98 |
| ATOM | 894 | O | ARG | 95 | 40.633 | 52.213 | 8.651 | 1.00 | 25.66 |
| ATOM | 895 | N | THR | 96 | 38.605 | 52.532 | 9.520 | 1.00 | 14.80 |
| ATOM | 898 | CB | THR | 96 | 39.459 | 51.542 | 11.670 | 1.00 | 36.32 |
| ATOM | 899 | OG1 | THR | 96 | 38.718 | 52.498 | 12.439 | 1.00 | 36.32 |
| ATOM | 901 | CG2 | THR | 96 | 40.908 | 52.045 | 11.476 | 1.00 | 36.32 |
| ATOM | 902 | C | THR | 96 | 37.365 | 50.730 | 10.607 | 1.00 | 14.80 |
| ATOM | 903 | O | THR | 96 | 36.340 | 51.326 | 10.292 | 1.00 | 36.32 |

TABLE 9
Three dimensional coordinates of
HC - CDR1 (ASN31-ASN35) from SB249417

| | | | | | | | | | |
|---|---|---|---|---|---|---|---|---|---|
| ATOM | 2300 | N | ASN | 31 | 53.647 | 23.490 | 34.881 | 1.00 | 20.53 |
| ATOM | 2302 | CA | ASN | 31 | 54.400 | 24.257 | 33.887 | 1.00 | 20.53 |
| ATOM | 2303 | CB | ASN | 31 | 53.820 | 25.666 | 33.715 | 1.00 | 39.50 |
| ATOM | 2304 | CG | ASN | 31 | 53.118 | 25.859 | 32.376 | 1.00 | 39.50 |
| ATOM | 2305 | OD1 | ASN | 31 | 53.469 | 25.236 | 31.370 | 1.00 | 39.50 |
| ATOM | 2306 | ND2 | ASN | 31 | 52.128 | 26.741 | 32.358 | 1.00 | 39.50 |
| ATOM | 2309 | C | ASN | 31 | 55.860 | 24.369 | 34.306 | 1.00 | 20.53 |
| ATOM | 2310 | O | ASN | 31 | 56.746 | 24.530 | 33.466 | 1.00 | 39.50 |
| ATOM | 2311 | N | TYR | 32 | 56.103 | 24.314 | 35.612 | 1.00 | 18.56 |
| ATOM | 2313 | CA | TYR | 32 | 57.458 | 24.408 | 36.148 | 1.00 | 18.56 |
| ATOM | 2314 | CB | TYR | 32 | 57.571 | 25.582 | 37.122 | 1.00 | 41.90 |
| ATOM | 2315 | CG | TYR | 32 | 57.374 | 26.943 | 36.499 | 1.00 | 41.90 |
| ATOM | 2316 | CD1 | TYR | 32 | 56.107 | 27.516 | 36.415 | 1.00 | 41.90 |
| ATOM | 2317 | CE1 | TYR | 32 | 55.923 | 28.782 | 35.869 | 1.00 | 41.90 |
| ATOM | 2318 | CD2 | TYR | 32 | 58.459 | 27.672 | 36.018 | 1.00 | 41.90 |
| ATOM | 2319 | CE2 | TYR | 32 | 58.288 | 28.940 | 35.472 | 1.00 | 41.90 |
| ATOM | 2320 | CZ | TYR | 32 | 57.017 | 29.489 | 35.402 | 1.00 | 41.90 |
| ATOM | 2321 | OH | TYR | 32 | 56.836 | 30.745 | 34.875 | 1.00 | 41.90 |
| ATOM | 2323 | C | TYR | 32 | 57.824 | 23.124 | 36.875 | 1.00 | 18.56 |
| ATOM | 2324 | O | TYR | 32 | 57.024 | 22.590 | 37.642 | 1.00 | 41.90 |
| ATOM | 2325 | N | GLY | 33 | 59.032 | 22.631 | 36.626 | 1.00 | 32.09 |
| ATOM | 2327 | CA | GLY | 33 | 59.480 | 21.415 | 37.276 | 1.00 | 32.09 |
| ATOM | 2328 | C | GLY | 33 | 59.805 | 21.659 | 38.736 | 1.00 | 32.09 |
| ATOM | 2329 | O | GLY | 33 | 60.028 | 22.802 | 39.140 | 1.00 | 20.56 |
| ATOM | 2330 | N | MET | 34 | 59.813 | 20.593 | 39.530 | 1.00 | 8.75 |
| ATOM | 2332 | CA | MET | 34 | 60.119 | 20.700 | 40.949 | 1.00 | 8.75 |
| ATOM | 2333 | CB | MET | 34 | 58.988 | 20.101 | 41.787 | 1.00 | 26.05 |
| ATOM | 2334 | CG | MET | 34 | 59.129 | 20.334 | 43.283 | 1.00 | 26.05 |
| ATOM | 2335 | SD | MET | 34 | 59.069 | 22.082 | 43.705 | 1.00 | 26.05 |
| ATOM | 2336 | CE | MET | 34 | 57.315 | 22.344 | 43.849 | 1.00 | 26.05 |
| ATOM | 2337 | C | MET | 34 | 61.417 | 19.972 | 41.256 | 1.00 | 8.75 |
| ATOM | 2338 | O | MET | 34 | 61.514 | 18.759 | 41.073 | 1.00 | 26.05 |
| ATOM | 2339 | N | ASN | 35 | 62.425 | 20.722 | 41.687 | 1.00 | 25.14 |
| ATOM | 2341 | CA | ASN | 35 | 63.720 | 20.147 | 42.034 | 1.00 | 25.14 |
| ATOM | 2342 | CB | ASN | 35 | 64.859 | 21.091 | 41.642 | 1.00 | 22.15 |
| ATOM | 2343 | CG | ASN | 35 | 65.135 | 21.097 | 40.156 | 1.00 | 22.15 |
| ATOM | 2344 | OD1 | ASN | 35 | 65.207 | 22.152 | 39.533 | 1.00 | 22.15 |
| ATOM | 2345 | ND2 | ASN | 35 | 65.347 | 19.921 | 39.588 | 1.00 | 22.15 |
| ATOM | 2348 | C | ASN | 35 | 63.785 | 19.906 | 43.533 | 1.00 | 25.14 |
| ATOM | 2349 | O | ASN | 35 | 63.256 | 20.693 | 44.316 | 1.00 | 22.15 |

TABLE 10
Three dimensional coordinates of
HC - CDR2 (TRP50-GLY66) from SB 249417

| | | | | | | | | | |
|---|---|---|---|---|---|---|---|---|---|
| ATOM | 2490 | N | TRP | 50 | 64.690 | 15.841 | 39.634 | 1.00 | 10.37 |
| ATOM | 2492 | CA | TRP | 50 | 63.706 | 16.753 | 39.073 | 1.00 | 10.37 |
| ATOM | 2493 | CB | TRP | 50 | 64.255 | 17.405 | 37.796 | 1.00 | 82.57 |
| ATOM | 2494 | CG | TRP | 50 | 64.648 | 16.439 | 36.697 | 1.00 | 82.57 |
| ATOM | 2495 | CD2 | TRP | 50 | 64.574 | 16.669 | 35.282 | 1.00 | 82.57 |
| ATOM | 2496 | CE2 | TRP | 50 | 65.053 | 15.504 | 34.643 | 1.00 | 82.57 |
| ATOM | 2497 | CE3 | TRP | 50 | 64.150 | 17.748 | 34.494 | 1.00 | 82.57 |
| ATOM | 2498 | CD1 | TRP | 50 | 65.155 | 15.177 | 36.849 | 1.00 | 82.57 |
| ATOM | 2499 | NE1 | TRP | 50 | 65.400 | 14.610 | 35.622 | 1.00 | 82.57 |
| ATOM | 2501 | CZ2 | TRP | 50 | 65.121 | 15.386 | 33.249 | 1.00 | 82.57 |
| ATOM | 2502 | CZ3 | TRP | 50 | 64.219 | 17.629 | 33.106 | 1.00 | 82.57 |
| ATOM | 2503 | CH2 | TRP | 50 | 64.701 | 16.456 | 32.501 | 1.00 | 82.57 |
| ATOM | 2504 | C | TRP | 50 | 62.412 | 16.021 | 38.760 | 1.00 | 10.37 |
| ATOM | 2505 | O | TRP | 50 | 62.403 | 14.800 | 38.616 | 1.00 | 82.57 |
| ATOM | 2506 | N | ILE | 51 | 61.315 | 16.766 | 38.728 | 1.00 | 26.53 |
| ATOM | 2508 | CA | ILE | 51 | 60.001 | 16.222 | 38.405 | 1.00 | 26.53 |
| ATOM | 2509 | CB | ILE | 51 | 59.025 | 16.284 | 39.603 | 1.00 | 25.59 |
| ATOM | 2510 | CG2 | ILE | 51 | 57.689 | 15.659 | 39.225 | 1.00 | 25.59 |
| ATOM | 2511 | CG1 | ILE | 51 | 59.599 | 15.545 | 40.810 | 1.00 | 25.59 |
| ATOM | 2512 | CD1 | ILE | 51 | 58.687 | 15.577 | 42.024 | 1.00 | 25.59 |
| ATOM | 2513 | C | ILE | 51 | 59.476 | 17.151 | 37.319 | 1.00 | 26.53 |
| ATOM | 2514 | O | ILE | 51 | 59.386 | 18.359 | 37.531 | 1.00 | 25.59 |
| ATOM | 2515 | N | ASN | 52 | 59.153 | 16.601 | 36.155 | 1.00 | 46.03 |
| ATOM | 2517 | CA | ASN | 52 | 58.651 | 17.415 | 35.047 | 1.00 | 46.03 |
| ATOM | 2518 | CB | ASN | 52 | 58.528 | 16.569 | 33.783 | 1.00 | 45.75 |
| ATOM | 2519 | CG | ASN | 52 | 58.447 | 17.406 | 32.528 | 1.00 | 45.75 |
| ATOM | 2520 | OD1 | ASN | 52 | 57.625 | 18.311 | 32.421 | 1.00 | 45.75 |
| ATOM | 2521 | ND2 | ASN | 52 | 59.298 | 17.097 | 31.561 | 1.00 | 45.75 |
| ATOM | 2524 | C | ASN | 52 | 57.300 | 18.040 | 35.377 | 1.00 | 46.03 |
| ATOM | 2525 | O | ASN | 52 | 56.899 | 19.032 | 34.768 | 1.00 | 45.75 |
| ATOM | 2526 | N | THR | 53 | 56.605 | 17.449 | 36.343 | 1.00 | 38.29 |
| ATOM | 2528 | CA | THR | 53 | 55.293 | 17.917 | 36.778 | 1.00 | 38.29 |
| ATOM | 2529 | CB | THR | 53 | 55.272 | 19.452 | 37.009 | 1.00 | 46.19 |
| ATOM | 2530 | OG1 | THR | 53 | 56.181 | 19.781 | 38.067 | 1.00 | 46.19 |
| ATOM | 2532 | CG2 | THR | 53 | 53.880 | 19.924 | 37.393 | 1.00 | 46.19 |
| ATOM | 2533 | C | THR | 53 | 54.194 | 17.476 | 35.812 | 1.00 | 38.29 |
| ATOM | 2534 | O | THR | 53 | 53.298 | 16.727 | 36.203 | 1.00 | 46.19 |
| ATOM | 2535 | N | ARG | 54 | 54.265 | 17.906 | 34.555 | 1.00 | 53.63 |
| ATOM | 2537 | CA | ARG | 54 | 53.261 | 17.500 | 33.573 | 1.00 | 53.63 |
| ATOM | 2538 | CB | ARG | 54 | 53.359 | 18.345 | 32.298 | 1.00 | 31.29 |
| ATOM | 2539 | CG | ARG | 54 | 54.717 | 18.334 | 31.631 | 1.00 | 31.29 |
| ATOM | 2540 | CD | ARG | 54 | 54.742 | 19.227 | 30.409 | 1.00 | 31.29 |
| ATOM | 2541 | NE | ARG | 54 | 56.062 | 19.229 | 29.782 | 1.00 | 31.29 |
| ATOM | 2543 | CZ | ARG | 54 | 56.666 | 20.315 | 29.312 | 1.00 | 31.29 |
| ATOM | 2544 | NH1 | ARG | 54 | 56.071 | 21.499 | 29.396 | 1.00 | 31.29 |
| ATOM | 2547 | NH2 | ARG | 54 | 57.871 | 20.218 | 28.766 | 1.00 | 31.29 |
| ATOM | 2550 | C | ARG | 54 | 53.457 | 16.013 | 33.267 | 1.00 | 53.63 |
| ATOM | 2551 | O | ARG | 54 | 54.507 | 15.603 | 32.771 | 1.00 | 31.29 |
| ATOM | 2552 | N | ASN | 55 | 52.477 | 15.208 | 33.670 | 1.00 | 58.18 |
| ATOM | 2554 | CA | ASN | 55 | 52.500 | 13.754 | 33.486 | 1.00 | 58.18 |
| ATOM | 2555 | CB | ASN | 55 | 52.879 | 13.373 | 32.044 | 1.00 | 44.20 |
| ATOM | 2556 | CG | ASN | 55 | 52.809 | 11.870 | 31.785 | 1.00 | 44.20 |
| ATOM | 2557 | OD1 | ASN | 55 | 53.602 | 11.326 | 31.017 | 1.00 | 44.20 |
| ATOM | 2558 | ND2 | ASN | 55 | 51.847 | 11.197 | 32.411 | 1.00 | 44.20 |
| ATOM | 2561 | C | ASN | 55 | 53.462 | 13.105 | 34.481 | 1.00 | 58.18 |
| ATOM | 2562 | O | ASN | 55 | 53.658 | 11.888 | 34.468 | 1.00 | 44.20 |
| ATOM | 2563 | N | GLY | 56 | 54.013 | 13.916 | 35.381 | 1.00 | 35.82 |
| ATOM | 2565 | CA | GLY | 56 | 54.947 | 13.406 | 36.370 | 1.00 | 35.82 |
| ATOM | 2566 | C | GLY | 56 | 56.103 | 12.672 | 35.723 | 1.00 | 35.82 |
| ATOM | 2567 | O | GLY | 56 | 56.637 | 11.715 | 36.281 | 1.00 | 33.62 |
| ATOM | 2568 | N | LYS | 57 | 56.477 | 13.118 | 34.529 | 1.00 | 56.46 |
| ATOM | 2570 | CA | LYS | 57 | 57.571 | 12.505 | 33.790 | 1.00 | 56.46 |
| ATOM | 2571 | CB | LYS | 57 | 57.305 | 12.584 | 32.281 | 1.00 | 42.16 |
| ATOM | 2572 | CG | LYS | 57 | 57.015 | 13.984 | 31.749 | 1.00 | 42.16 |
| ATOM | 2573 | CD | LYS | 57 | 56.585 | 13.927 | 30.289 | 1.00 | 42.16 |
| ATOM | 2574 | CE | LYS | 57 | 56.184 | 15.294 | 29.747 | 1.00 | 42.16 |
| ATOM | 2575 | NZ | LYS | 57 | 57.344 | 16.189 | 29.495 | 1.00 | 42.16 |
| ATOM | 2579 | C | LYS | 57 | 58.900 | 13.160 | 34.138 | 1.00 | 56.46 |
| ATOM | 2580 | O | LYS | 57 | 58.987 | 13.933 | 35.098 | 1.00 | 42.16 |
| ATOM | 2581 | N | SER | 58 | 59.930 | 12.832 | 33.361 | 1.00 | 69.70 |
| ATOM | 2583 | CA | SER | 58 | 61.273 | 13.374 | 33.548 | 1.00 | 69.70 |
| ATOM | 2584 | CB | SER | 58 | 61.377 | 14.767 | 32.920 | 1.00 | 51.34 |
| ATOM | 2585 | OG | SER | 58 | 61.034 | 14.740 | 31.541 | 1.00 | 51.34 |
| ATOM | 2587 | C | SER | 58 | 61.679 | 13.421 | 35.016 | 1.00 | 69.70 |
| ATOM | 2588 | O | SER | 58 | 61.711 | 14.489 | 35.631 | 1.00 | 51.34 |
| ATOM | 2589 | N | THR | 59 | 61.928 | 12.245 | 35.578 | 1.00 | 66.55 |
| ATOM | 2591 | CA | THR | 59 | 62.336 | 12.118 | 36.969 | 1.00 | 66.55 |
| ATOM | 2592 | CB | THR | 59 | 61.465 | 11.076 | 37.702 | 1.00 | 41.34 |
| ATOM | 2593 | OG1 | THR | 59 | 60.280 | 10.821 | 36.937 | 1.00 | 41.34 |
| ATOM | 2595 | CG2 | THR | 59 | 61.058 | 11.594 | 39.066 | 1.00 | 41.34 |
| ATOM | 2596 | C | THR | 59 | 63.774 | 11.622 | 36.924 | 1.00 | 66.55 |
| ATOM | 2597 | O | THR | 59 | 64.129 | 10.848 | 36.029 | 1.00 | 41.34 |
| ATOM | 2598 | N | TYR | 60 | 64.621 | 12.091 | 37.835 | 1.00 | 40.30 |
| ATOM | 2600 | CA | TYR | 60 | 66.002 | 11.629 | 37.823 | 1.00 | 40.30 |
| ATOM | 2601 | CB | TYR | 60 | 66.869 | 12.381 | 38.835 | 1.00 | 68.57 |
| ATOM | 2602 | CG | TYR | 60 | 68.285 | 11.842 | 38.911 | 1.00 | 68.57 |
| ATOM | 2603 | CD1 | TYR | 60 | 68.980 | 11.483 | 37.755 | 1.00 | 68.57 |
| ATOM | 2604 | CE1 | TYR | 60 | 70.255 | 10.929 | 37.821 | 1.00 | 68.57 |
| ATOM | 2605 | CD2 | TYR | 60 | 68.910 | 11.639 | 40.137 | 1.00 | 68.57 |
| ATOM | 2606 | CE2 | TYR | 60 | 70.186 | 11.087 | 40.214 | 1.00 | 68.57 |
| ATOM | 2607 | CZ | TYR | 60 | 70.852 | 10.734 | 39.055 | 1.00 | 68.57 |
| ATOM | 2608 | OH | TYR | 60 | 72.108 | 10.181 | 39.136 | 1.00 | 68.57 |
| ATOM | 2610 | C | TYR | 60 | 66.035 | 10.136 | 38.119 | 1.00 | 40.30 |
| ATOM | 2611 | O | TYR | 60 | 65.463 | 9.683 | 39.106 | 1.00 | 68.57 |
| ATOM | 2612 | N | VAL | 61 | 66.720 | 9.387 | 37.258 | 1.00 | 78.68 |
| ATOM | 2614 | CA | VAL | 61 | 66.857 | 7.935 | 37.386 | 1.00 | 78.68 |
| ATOM | 2615 | CB | VAL | 61 | 67.864 | 7.381 | 36.341 | 1.00 | 61.99 |
| ATOM | 2616 | CG1 | VAL | 61 | 67.881 | 5.852 | 36.363 | 1.00 | 61.99 |
| ATOM | 2617 | CG2 | VAL | 61 | 67.518 | 7.891 | 34.945 | 1.00 | 61.99 |
| ATOM | 2618 | C | VAL | 61 | 67.323 | 7.531 | 38.788 | 1.00 | 78.68 |
| ATOM | 2619 | O | VAL | 61 | 67.113 | 6.396 | 39.218 | 1.00 | 61.99 |
| ATOM | 2620 | N | ASP | 62 | 67.955 | 8.468 | 39.491 | 1.00 | 56.17 |
| ATOM | 2622 | CA | ASP | 62 | 68.455 | 8.234 | 40.840 | 1.00 | 56.17 |
| ATOM | 2623 | CB | ASP | 62 | 67.298 | 7.887 | 41.784 | 1.00 | 45.87 |
| ATOM | 2624 | CG | ASP | 62 | 66.192 | 8.938 | 41.764 | 1.00 | 45.87 |
| ATOM | 2625 | OD1 | ASP | 62 | 66.499 | 10.131 | 41.559 | 1.00 | 45.87 |
| ATOM | 2626 | OD2 | ASP | 62 | 65.009 | 8.573 | 41.936 | 1.00 | 45.87 |
| ATOM | 2627 | C | ASP | 62 | 69.511 | 7.134 | 40.810 | 1.00 | 56.17 |
| ATOM | 2628 | O | ASP | 62 | 69.207 | 5.953 | 40.977 | 1.00 | 45.87 |
| ATOM | 2629 | N | ASP | 63 | 70.755 | 7.543 | 40.574 | 1.00 | 73.06 |
| ATOM | 2631 | CA | ASP | 63 | 71.885 | 6.623 | 40.492 | 1.00 | 73.06 |
| ATOM | 2632 | CB | ASP | 63 | 73.194 | 7.404 | 40.344 | 1.00 | 43.18 |
| ATOM | 2633 | CG | ASP | 63 | 73.946 | 7.051 | 39.072 | 1.00 | 43.18 |
| ATOM | 2634 | OD1 | ASP | 63 | 73.828 | 5.897 | 38.604 | 1.00 | 43.18 |
| ATOM | 2635 | OD2 | ASP | 63 | 74.667 | 7.924 | 38.546 | 1.00 | 43.18 |
| ATOM | 2636 | C | ASP | 63 | 71.972 | 5.697 | 41.696 | 1.00 | 73.06 |
| ATOM | 2637 | O | ASP | 63 | 72.399 | 6.110 | 42.776 | 1.00 | 43.18 |
| ATOM | 2638 | N | PHE | 64 | 71.509 | 4.461 | 41.515 | 1.00 | 77.35 |
| ATOM | 2640 | CA | PHE | 64 | 71.521 | 3.437 | 42.562 | 1.00 | 77.35 |
| ATOM | 2641 | CB | PHE | 64 | 72.948 | 3.202 | 43.071 | 1.00 | 68.41 |
| ATOM | 2642 | CG | PHE | 64 | 73.486 | 1.836 | 42.762 | 1.00 | 68.41 |
| ATOM | 2643 | CD1 | PHE | 64 | 73.432 | 1.328 | 41.467 | 1.00 | 68.41 |
| ATOM | 2644 | CD2 | PHE | 64 | 74.047 | 1.053 | 43.766 | 1.00 | 68.41 |
| ATOM | 2645 | CE1 | PHE | 64 | 73.930 | 0.058 | 41.177 | 1.00 | 68.41 |
| ATOM | 2646 | CE2 | PHE | 64 | 74.548 | −0.219 | 43.485 | 1.00 | 68.41 |
| ATOM | 2647 | CZ | PHE | 64 | 74.489 | −0.717 | 42.188 | 1.00 | 68.41 |
| ATOM | 2648 | C | PHE | 64 | 70.592 | 3.723 | 43.740 | 1.00 | 77.35 |
| ATOM | 2649 | O | PHE | 64 | 70.141 | 2.795 | 44.419 | 1.00 | 68.41 |
| ATOM | 2650 | N | LYS | 65 | 70.284 | 4.996 | 43.967 | 1.00 | 77.80 |
| ATOM | 2652 | CA | LYS | 65 | 69.414 | 5.395 | 45.066 | 1.00 | 77.80 |
| ATOM | 2653 | CB | LYS | 65 | 69.749 | 6.824 | 45.525 | 1.00 | 59.28 |
| ATOM | 2654 | CG | LYS | 65 | 71.243 | 7.133 | 45.654 | 1.00 | 59.28 |
| ATOM | 2655 | CD | LYS | 65 | 72.017 | 6.042 | 46.394 | 1.00 | 59.28 |
| ATOM | 2656 | CE | LYS | 65 | 71.576 | 5.890 | 47.841 | 1.00 | 59.28 |
| ATOM | 2657 | NZ | LYS | 65 | 72.374 | 4.831 | 48.536 | 1.00 | 59.28 |
| ATOM | 2661 | C | LYS | 65 | 67.940 | 5.296 | 44.675 | 1.00 | 77.80 |
| ATOM | 2662 | O | LYS | 65 | 67.188 | 6.271 | 44.781 | 1.00 | 59.28 |
| ATOM | 2663 | N | GLY | 66 | 67.523 | 4.105 | 44.255 | 1.00 | 33.49 |
| ATOM | 2665 | CA | GLY | 66 | 66.141 | 3.893 | 43.863 | 1.00 | 33.49 |
| ATOM | 2666 | C | GLY | 66 | 65.248 | 3.671 | 45.067 | 1.00 | 33.49 |
| ATOM | 2667 | O | GLY | 66 | 64.479 | 2.710 | 45.117 | 1.00 | 26.03 |

TABLE 11
Three dimensional coordinates of
HC - CDR3 (GLU99-TYR110) from SB249417

| | | | | | | | | | |
|---|---|---|---|---|---|---|---|---|---|
| ATOM | 2507 | N | GLU | 99 | 61.719 | 25.581 | 38.831 | 1.00 | 50.46 |
| ATOM | 2508 | CA | GLU | 99 | 62.445 | 25.725 | 37.560 | 1.00 | 50.46 |
| ATOM | 2509 | CB | GLU | 99 | 63.093 | 27.110 | 37.435 | 1.00 | 52.10 |
| ATOM | 2510 | CG | GLU | 99 | 62.109 | 28.216 | 37.059 | 1.00 | 52.10 |
| ATOM | 2511 | CD | GLU | 99 | 62.112 | 29.390 | 38.028 | 1.00 | 52.10 |
| ATOM | 2512 | OE1 | GLU | 99 | 61.436 | 30.397 | 37.735 | 1.00 | 52.10 |
| ATOM | 2513 | OE2 | GLU | 99 | 62.772 | 29.310 | 39.086 | 1.00 | 52.10 |
| ATOM | 2514 | C | GLU | 99 | 63.461 | 24.618 | 37.297 | 1.00 | 50.46 |
| ATOM | 2515 | O | GLU | 99 | 63.484 | 23.616 | 38.010 | 1.00 | 52.10 |
| ATOM | 2516 | N | GLY | 100 | 64.259 | 24.775 | 36.242 | 1.00 | 42.17 |
| ATOM | 2517 | CA | GLY | 100 | 65.249 | 23.764 | 35.914 | 1.00 | 42.17 |
| ATOM | 2518 | C | GLY | 100 | 66.331 | 24.192 | 34.937 | 1.00 | 42.17 |
| ATOM | 2519 | O | GLY | 100 | 66.089 | 24.997 | 34.033 | 1.00 | 27.11 |
| ATOM | 2520 | N | ASN | 101 | 67.526 | 23.635 | 35.132 | 1.00 | 59.61 |
| ATOM | 2521 | CA | ASN | 101 | 68.704 | 23.902 | 34.306 | 1.00 | 59.61 |
| ATOM | 2522 | CB | ASN | 101 | 68.654 | 23.089 | 33.006 | 1.00 | 55.09 |
| ATOM | 2523 | CG | ASN | 101 | 68.926 | 21.612 | 33.229 | 1.00 | 55.09 |
| ATOM | 2524 | OD1 | ASN | 101 | 68.323 | 20.985 | 34.102 | 1.00 | 55.09 |
| ATOM | 2525 | ND2 | ASN | 101 | 69.834 | 21.046 | 32.439 | 1.00 | 55.09 |
| ATOM | 2528 | C | ASN | 101 | 68.940 | 25.379 | 34.011 | 1.00 | 59.61 |
| ATOM | 2529 | O | ASN | 101 | 69.643 | 26.062 | 34.763 | 1.00 | 55.09 |
| ATOM | 2530 | N | MET | 102 | 68.369 | 25.867 | 32.914 | 1.00 | 51.25 |
| ATOM | 2531 | CA | MET | 102 | 68.514 | 27.265 | 32.530 | 1.00 | 51.25 |
| ATOM | 2532 | CB | MET | 102 | 69.931 | 27.556 | 32.037 | 1.00 | 39.15 |
| ATOM | 2533 | CG | MET | 102 | 70.367 | 29.002 | 32.229 | 1.00 | 39.15 |
| ATOM | 2534 | SD | MET | 102 | 69.099 | 30.248 | 31.922 | 1.00 | 39.15 |
| ATOM | 2535 | CE | MET | 102 | 69.132 | 31.094 | 33.482 | 1.00 | 39.15 |
| ATOM | 2536 | C | MET | 102 | 67.519 | 27.571 | 31.424 | 1.00 | 51.25 |
| ATOM | 2537 | O | MET | 102 | 67.866 | 27.577 | 30.241 | 1.00 | 39.15 |
| ATOM | 2538 | N | ASP | 103 | 66.270 | 27.787 | 31.814 | 1.00 | 52.39 |
| ATOM | 2539 | CA | ASP | 103 | 65.210 | 28.095 | 30.867 | 1.00 | 52.39 |
| ATOM | 2540 | CB | ASP | 103 | 64.309 | 26.865 | 30.664 | 1.00 | 81.62 |
| ATOM | 2541 | CG | ASP | 103 | 65.099 | 25.592 | 30.350 | 1.00 | 81.62 |
| ATOM | 2542 | OD1 | ASP | 103 | 64.784 | 24.533 | 30.939 | 1.00 | 81.62 |
| ATOM | 2543 | OD2 | ASP | 103 | 66.028 | 25.642 | 29.514 | 1.00 | 81.62 |
| ATOM | 2544 | C | ASP | 103 | 64.391 | 29.250 | 31.440 | 1.00 | 52.39 |
| ATOM | 2545 | O | ASP | 103 | 64.181 | 29.324 | 32.653 | 1.00 | 81.62 |
| ATOM | 2546 | N | GLY | 104 | 63.980 | 30.176 | 30.577 | 1.00 | 38.36 |
| ATOM | 2547 | CA | GLY | 104 | 63.181 | 31.309 | 31.019 | 1.00 | 38.36 |
| ATOM | 2548 | C | GLY | 104 | 63.874 | 32.286 | 31.954 | 1.00 | 38.36 |
| ATOM | 2549 | O | GLY | 104 | 63.209 | 33.068 | 32.630 | 1.00 | 35.81 |
| ATOM | 2550 | N | TYR | 105 | 65.204 | 32.221 | 32.005 | 1.00 | 78.70 |
| ATOM | 2551 | CA | TYR | 105 | 66.028 | 33.098 | 32.843 | 1.00 | 78.70 |
| ATOM | 2552 | CB | TYR | 105 | 66.298 | 34.426 | 32.125 | 1.00 | 41.71 |
| ATOM | 2553 | CG | TYR | 105 | 67.726 | 34.593 | 31.653 | 1.00 | 41.71 |
| ATOM | 2554 | CD1 | TYR | 105 | 68.492 | 33.493 | 31.266 | 1.00 | 41.71 |
| ATOM | 2555 | CE1 | TYR | 105 | 69.812 | 33.644 | 30.838 | 1.00 | 41.71 |
| ATOM | 2556 | CD2 | TYR | 105 | 68.315 | 35.854 | 31.599 | 1.00 | 41.71 |
| ATOM | 2557 | CE2 | TYR | 105 | 69.632 | 36.017 | 31.172 | 1.00 | 41.71 |
| ATOM | 2558 | CZ | TYR | 105 | 70.372 | 34.910 | 30.794 | 1.00 | 41.71 |
| ATOM | 2559 | OH | TYR | 105 | 71.671 | 35.077 | 30.382 | 1.00 | 41.71 |
| ATOM | 2560 | C | TYR | 105 | 65.515 | 33.355 | 34.263 | 1.00 | 78.70 |
| ATOM | 2561 | O | TYR | 105 | 65.349 | 34.503 | 34.679 | 1.00 | 41.71 |
| ATOM | 2562 | N | PHE | 106 | 65.297 | 32.275 | 35.006 | 1.00 | 53.79 |
| ATOM | 2563 | CA | PHE | 106 | 64.815 | 32.362 | 36.381 | 1.00 | 53.79 |
| ATOM | 2564 | CB | PHE | 106 | 63.302 | 32.606 | 36.355 | 1.00 | 64.88 |
| ATOM | 2565 | CG | PHE | 106 | 62.867 | 33.837 | 37.093 | 1.00 | 64.88 |
| ATOM | 2566 | CD1 | PHE | 106 | 63.142 | 35.104 | 36.586 | 1.00 | 64.88 |
| ATOM | 2567 | CD2 | PHE | 106 | 62.162 | 33.732 | 38.286 | 1.00 | 64.88 |
| ATOM | 2568 | CE1 | PHE | 106 | 62.722 | 36.246 | 37.255 | 1.00 | 64.88 |
| ATOM | 2569 | CE2 | PHE | 106 | 61.736 | 34.868 | 38.964 | 1.00 | 64.88 |
| ATOM | 2570 | CZ | PHE | 106 | 62.016 | 36.129 | 38.447 | 1.00 | 64.88 |
| ATOM | 2571 | C | PHE | 106 | 65.099 | 31.140 | 37.282 | 1.00 | 53.79 |
| ATOM | 2572 | O | PHE | 106 | 64.552 | 31.055 | 38.381 | 1.00 | 64.88 |
| ATOM | 2573 | N | PRO | 107 | 66.005 | 30.221 | 36.878 | 1.00 | 52.80 |
| ATOM | 2574 | CD | PRO | 107 | 66.836 | 30.142 | 35.667 | 1.00 | 42.34 |
| ATOM | 2575 | CA | PRO | 107 | 66.268 | 29.051 | 37.725 | 1.00 | 52.80 |
| ATOM | 2576 | CB | PRO | 107 | 67.393 | 28.338 | 36.977 | 1.00 | 42.34 |
| ATOM | 2577 | CG | PRO | 107 | 67.103 | 28.666 | 35.568 | 1.00 | 42.34 |
| ATOM | 2578 | C | PRO | 107 | 66.623 | 29.241 | 39.198 | 1.00 | 52.80 |
| ATOM | 2579 | O | PRO | 107 | 67.275 | 30.212 | 39.596 | 1.00 | 42.34 |
| ATOM | 2580 | N | PHE | 108 | 66.199 | 28.252 | 39.980 | 1.00 | 39.89 |
| ATOM | 2581 | CA | PHE | 108 | 66.421 | 28.160 | 41.417 | 1.00 | 39.89 |
| ATOM | 2582 | CB | PHE | 108 | 67.823 | 27.639 | 41.713 | 1.00 | 29.53 |
| ATOM | 2583 | CG | PHE | 108 | 67.986 | 26.177 | 41.417 | 1.00 | 29.53 |
| ATOM | 2584 | CD1 | PHE | 108 | 67.950 | 25.711 | 40.107 | 1.00 | 29.53 |
| ATOM | 2585 | CD2 | PHE | 108 | 68.127 | 25.258 | 42.450 | 1.00 | 29.53 |
| ATOM | 2586 | CE1 | PHE | 108 | 68.049 | 24.350 | 39.833 | 1.00 | 29.53 |
| ATOM | 2587 | CE2 | PHE | 108 | 68.228 | 23.894 | 42.186 | 1.00 | 29.53 |
| ATOM | 2588 | CZ | PHE | 108 | 68.188 | 23.440 | 40.878 | 1.00 | 29.53 |
| ATOM | 2589 | C | PHE | 108 | 66.057 | 29.348 | 42.287 | 1.00 | 39.89 |
| ATOM | 2590 | O | PHE | 108 | 66.654 | 29.578 | 43.342 | 1.00 | 29.53 |
| ATOM | 2591 | N | THR | 109 | 65.082 | 30.115 | 41.821 | 1.00 | 37.60 |
| ATOM | 2592 | CA | THR | 109 | 64.572 | 31.243 | 42.571 | 1.00 | 37.60 |
| ATOM | 2593 | CB | THR | 109 | 64.110 | 32.374 | 41.638 | 1.00 | 39.99 |
| ATOM | 2594 | OG1 | THR | 109 | 63.235 | 31.842 | 40.638 | 1.00 | 39.99 |
| ATOM | 2595 | CG2 | THR | 109 | 65.303 | 33.016 | 40.950 | 1.00 | 39.99 |
| ATOM | 2596 | C | THR | 109 | 63.369 | 30.609 | 43.267 | 1.00 | 37.60 |
| ATOM | 2597 | O | THR | 109 | 62.694 | 29.761 | 42.676 | 1.00 | 39.99 |
| ATOM | 2598 | N | TYR | 110 | 63.113 | 30.999 | 44.511 | 1.00 | 23.43 |
| ATOM | 2599 | CA | TYR | 110 | 62.006 | 30.449 | 45.292 | 1.00 | 23.43 |
| ATOM | 2600 | CB | TYR | 110 | 60.701 | 30.367 | 44.481 | 1.00 | 42.41 |
| ATOM | 2601 | CG | TYR | 110 | 60.156 | 31.673 | 43.951 | 1.00 | 42.41 |
| ATOM | 2602 | CD1 | TYR | 110 | 60.138 | 31.931 | 42.583 | 1.00 | 42.41 |
| ATOM | 2603 | CE1 | TYR | 110 | 59.587 | 33.104 | 42.077 | 1.00 | 42.41 |
| ATOM | 2504 | CD2 | TYR | 110 | 59.611 | 32.628 | 44.807 | 1.00 | 42.41 |
| ATOM | 2605 | CE2 | TYR | 110 | 59.055 | 33.807 | 44.309 | 1.00 | 42.41 |
| ATOM | 2606 | CZ | TYR | 110 | 59.047 | 34.035 | 42.942 | 1.00 | 42.41 |
| ATOM | 2607 | OH | TYR | 110 | 58.484 | 35.185 | 42.439 | 1.00 | 42.41 |
| ATOM | 2608 | C | TYR | 110 | 62.358 | 29.042 | 45.763 | 1.00 | 23.43 |
| ATOM | 2609 | O | TYR | 110 | 62.436 | 28.111 | 44.960 | 1.00 | 42.41 |

TABLE 12
Three dimensional coordinates of
LC - CDR1 (ARG24-HIS33) from SB249417

| | | | | | | | | | |
|---|---|---|---|---|---|---|---|---|---|
| ATOM | 161 | N | ARG | 24 | 85.923 | 25.430 | 39.568 | 1.00 | 40.61 |
| ATOM | 162 | CA | ARG | 24 | 86.364 | 24.572 | 38.468 | 1.00 | 40.61 |
| ATOM | 163 | CB | ARG | 24 | 87.477 | 23.636 | 38.953 | 1.00 | 54.47 |
| ATOM | 164 | CG | ARG | 24 | 88.672 | 23.525 | 38.021 | 1.00 | 54.47 |
| ATOM | 165 | CD | ARG | 24 | 89.786 | 24.476 | 38.433 | 1.00 | 54.47 |
| ATOM | 166 | NE | ARG | 24 | 89.329 | 25.861 | 38.511 | 1.00 | 54.47 |
| ATOM | 167 | CZ | ARG | 24 | 90.019 | 26.850 | 39.069 | 1.00 | 54.47 |
| ATOM | 168 | NH1 | ARG | 24 | 91.212 | 26.619 | 39.605 | 1.00 | 54.47 |
| ATOM | 171 | NH2 | ARG | 24 | 89.510 | 28.073 | 39.101 | 1.00 | 54.47 |
| ATOM | 174 | C | ARG | 24 | 85.191 | 23.729 | 37.974 | 1.00 | 40.61 |
| ATOM | 175 | O | ARG | 24 | 84.258 | 23.455 | 38.735 | 1.00 | 54.47 |
| ATOM | 176 | N | ALA | 25 | 85.251 | 23.296 | 36.718 | 1.00 | 33.05 |
| ATOM | 177 | CA | ALA | 25 | 84.185 | 22.475 | 36.146 | 1.00 | 33.05 |
| ATOM | 178 | CB | ALA | 25 | 83.270 | 23.332 | 35.275 | 1.00 | 58.44 |
| ATOM | 179 | C | ALA | 25 | 84.702 | 21.278 | 35.348 | 1.00 | 33.05 |
| ATOM | 180 | O | ALA | 25 | 83.923 | 20.409 | 34.958 | 1.00 | 58.44 |
| ATOM | 181 | N | SER | 26 | 86.006 | 21.249 | 35.037 | 1.00 | 57.44 |
| ATOM | 182 | CA | SER | 26 | 86.641 | 20.165 | 34.330 | 1.00 | 57.44 |
| ATOM | 183 | CB | SER | 26 | 86.518 | 18.828 | 35.080 | 1.00 | 65.59 |
| ATOM | 184 | OG | SER | 26 | 87.351 | 17.828 | 34.505 | 1.00 | 65.59 |
| ATOM | 185 | C | SER | 26 | 86.093 | 20.030 | 32.903 | 1.00 | 57.44 |
| ATOM | 186 | O | SER | 26 | 86.698 | 20.533 | 31.952 | 1.00 | 65.59 |
| ATOM | 187 | N | SER | 27 | 84.946 | 19.366 | 32.762 | 1.00 | 55.02 |
| ATOM | 188 | CA | SER | 27 | 84.317 | 19.158 | 31.459 | 1.00 | 55.02 |
| ATOM | 189 | CB | SER | 27 | 82.987 | 18.420 | 31.627 | 1.00 | 53.39 |
| ATOM | 190 | OG | SER | 27 | 83.183 | 17.167 | 32.259 | 1.00 | 53.39 |
| ATOM | 191 | C | SER | 27 | 84.091 | 20.476 | 30.725 | 1.00 | 55.02 |
| ATOM | 192 | O | SER | 27 | 84.718 | 20.717 | 29.690 | 1.00 | 53.39 |
| ATOM | 193 | N | SER | 28 | 83.232 | 21.316 | 31.307 | 1.00 | 33.90 |
| ATOM | 194 | CA | SER | 28 | 82.834 | 22.647 | 30.825 | 1.00 | 33.90 |
| ATOM | 195 | CB | SER | 28 | 83.830 | 23.274 | 29.833 | 1.00 | 57.68 |
| ATOM | 196 | OG | SER | 28 | 83.804 | 22.660 | 28.552 | 1.00 | 57.68 |
| ATOM | 197 | C | SER | 28 | 81.430 | 22.670 | 30.238 | 1.00 | 33.90 |
| ATOM | 198 | O | SER | 28 | 81.089 | 21.866 | 29.368 | 1.00 | 57.68 |
| ATOM | 199 | N | VAL | 29 | 80.619 | 23.592 | 30.742 | 1.00 | 39.14 |
| ATOM | 200 | CA | VAL | 29 | 79.244 | 23.773 | 30.294 | 1.00 | 39.14 |
| ATOM | 201 | CB | VAL | 29 | 78.226 | 23.123 | 31.278 | 1.00 | 50.11 |
| ATOM | 202 | CG1 | VAL | 29 | 78.394 | 21.612 | 31.295 | 1.00 | 50.11 |
| ATOM | 203 | CG2 | VAL | 29 | 78.401 | 23.688 | 32.681 | 1.00 | 50.11 |
| ATOM | 204 | C | VAL | 29 | 79.031 | 25.282 | 30.251 | 1.00 | 39.14 |
| ATOM | 205 | O | VAL | 29 | 79.981 | 26.036 | 30.028 | 1.00 | 50.11 |
| ATOM | 206 | N | ASN | 30 | 77.798 | 25.731 | 30.449 | 1.00 | 34.36 |
| ATOM | 207 | CA | ASN | 30 | 77.518 | 27.157 | 30.446 | 1.00 | 34.36 |
| ATOM | 208 | CB | ASN | 30 | 77.105 | 27.633 | 29.051 | 1.00 | 69.95 |
| ATOM | 209 | CG | ASN | 30 | 77.315 | 29.129 | 28.859 | 1.00 | 69.95 |
| ATOM | 210 | OD1 | ASN | 30 | 76.945 | 29.938 | 29.712 | 1.00 | 69.95 |
| ATOM | 211 | ND2 | ASN | 30 | 77.935 | 29.501 | 27.744 | 1.00 | 69.95 |
| ATOM | 214 | C | ASN | 30 | 76.405 | 27.416 | 31.437 | 1.00 | 34.36 |
| ATOM | 215 | O | ASN | 30 | 75.668 | 26.496 | 31.799 | 1.00 | 69.95 |
| ATOM | 216 | N | TYR | 31 | 76.313 | 28.662 | 31.895 | 1.00 | 51.94 |
| ATOM | 217 | CA | TYR | 31 | 75.299 | 29.094 | 32.853 | 1.00 | 51.94 |
| ATOM | 218 | CB | TYR | 31 | 73.896 | 28.690 | 32.379 | 1.00 | 66.29 |
| ATOM | 219 | CG | TYR | 31 | 73.464 | 29.386 | 31.105 | 1.00 | 66.29 |
| ATOM | 220 | CD1 | TYR | 31 | 72.980 | 28.661 | 30.016 | 1.00 | 66.29 |
| ATOM | 221 | CE1 | TYR | 31 | 72.567 | 29.305 | 28.844 | 1.00 | 66.29 |
| ATOM | 222 | CD2 | TYR | 31 | 73.528 | 30.773 | 30.993 | 1.00 | 66.29 |
| ATOM | 223 | CE2 | TYR | 31 | 73.120 | 31.424 | 29.832 | 1.00 | 66.29 |
| ATOM | 224 | CZ | TYR | 31 | 72.641 | 30.687 | 28.763 | 1.00 | 66.29 |
| ATOM | 225 | OH | TYR | 31 | 72.237 | 31.345 | 27.626 | 1.00 | 66.29 |
| ATOM | 226 | C | TYR | 31 | 75.562 | 28.609 | 34.276 | 1.00 | 51.94 |
| ATOM | 227 | O | TYR | 31 | 74.995 | 27.610 | 34.729 | 1.00 | 66.29 |
| ATOM | 228 | N | MET | 32 | 76.435 | 29.331 | 34.972 | 1.00 | 31.75 |
| ATOM | 229 | CA | MET | 32 | 76.788 | 29.013 | 36.351 | 1.00 | 31.75 |
| ATOM | 230 | CB | MET | 32 | 78.246 | 29.392 | 36.631 | 1.00 | 29.56 |
| ATOM | 231 | CG | MET | 32 | 78.807 | 28.822 | 37.925 | 1.00 | 29.56 |
| ATOM | 232 | SD | MET | 32 | 78.874 | 27.021 | 37.900 | 1.00 | 29.56 |
| ATOM | 233 | CE | MET | 32 | 80.515 | 26.716 | 38.506 | 1.00 | 29.56 |
| ATOM | 234 | C | MET | 32 | 75.857 | 29.820 | 37.246 | 1.00 | 31.75 |
| ATOM | 235 | O | MET | 32 | 75.576 | 30.984 | 36.960 | 1.00 | 29.56 |
| ATOM | 236 | N | HIS | 33 | 75.355 | 29.192 | 38.303 | 1.00 | 18.73 |
| ATOM | 237 | CA | HIS | 33 | 74.441 | 29.848 | 39.231 | 1.00 | 18.73 |
| ATOM | 238 | CB | HIS | 33 | 73.154 | 29.022 | 39.412 | 1.00 | 59.11 |
| ATOM | 239 | CG | HIS | 33 | 72.630 | 28.395 | 38.153 | 1.00 | 59.11 |
| ATOM | 240 | CD2 | HIS | 33 | 73.216 | 27.574 | 37.249 | 1.00 | 59.11 |
| ATOM | 241 | ND1 | HIS | 33 | 71.325 | 28.548 | 37.736 | 1.00 | 59.11 |
| ATOM | 242 | CE1 | HIS | 33 | 71.130 | 27.850 | 36.631 | 1.00 | 59.11 |
| ATOM | 243 | NE2 | HIS | 33 | 72.262 | 27.250 | 36.315 | 1.00 | 59.11 |
| ATOM | 244 | C | HIS | 33 | 75.136 | 29.943 | 40.584 | 1.00 | 18.73 |
| ATOM | 245 | O | HIS | 33 | 75.667 | 28.945 | 41.071 | 1.00 | 59.11 |

TABLE 13
Three dimensional coordinates of
LC - CDR2 (ALA49-SER55) from SB249417

| | | | | | | | | | |
|---|---|---|---|---|---|---|---|---|---|
| ATOM | 385 | N | ALA | 49 | 73.341 | 32.762 | 35.709 | 1.00 | 21.70 |
| ATOM | 386 | CA | ALA | 49 | 73.888 | 32.759 | 34.358 | 1.00 | 21.70 |
| ATOM | 387 | CB | ALA | 49 | 72.879 | 33.352 | 33.379 | 1.00 | 50.17 |
| ATOM | 388 | C | ALA | 49 | 75.206 | 33.523 | 34.298 | 1.00 | 21.70 |
| ATOM | 389 | O | ALA | 49 | 75.335 | 34.507 | 33.564 | 1.00 | 50.17 |
| ATOM | 390 | N | THR | 50 | 76.154 | 33.083 | 35.119 | 1.00 | 36.52 |
| ATOM | 391 | CA | THR | 50 | 77.494 | 33.655 | 35.211 | 1.00 | 36.52 |
| ATOM | 392 | CB | THR | 50 | 78.362 | 33.294 | 33.978 | 1.00 | 58.15 |
| ATOM | 393 | OG1 | THR | 50 | 77.656 | 33.610 | 32.773 | 1.00 | 58.15 |
| ATOM | 394 | CG2 | THR | 50 | 78.692 | 31.819 | 33.979 | 1.00 | 58.15 |
| ATOM | 395 | C | THR | 50 | 77.605 | 35.152 | 35.482 | 1.00 | 36.52 |
| ATOM | 396 | O | THR | 50 | 77.942 | 35.558 | 36.594 | 1.00 | 58.15 |
| ATOM | 397 | N | SER | 51 | 77.327 | 35.965 | 34.471 | 1.00 | 31.35 |
| ATOM | 398 | CA | SER | 51 | 77.441 | 37.413 | 34.592 | 1.00 | 31.35 |
| ATOM | 399 | CB | SER | 51 | 77.862 | 38.009 | 33.245 | 1.00 | 35.38 |
| ATOM | 400 | OG | SER | 51 | 79.126 | 37.511 | 32.836 | 1.00 | 35.38 |
| ATOM | 401 | C | SER | 51 | 76.228 | 38.169 | 35.120 | 1.00 | 31.35 |
| ATOM | 402 | O | SER | 51 | 76.352 | 39.325 | 35.528 | 1.00 | 35.38 |
| ATOM | 403 | N | ASN | 52 | 75.060 | 37.540 | 35.129 | 1.00 | 34.69 |
| ATOM | 404 | CA | ASN | 52 | 73.863 | 38.238 | 35.592 | 1.00 | 34.69 |
| ATOM | 405 | CB | ASN | 52 | 72.614 | 37.715 | 34.879 | 1.00 | 34.18 |
| ATOM | 406 | CG | ASN | 52 | 72.561 | 38.131 | 33.420 | 1.00 | 34.18 |
| ATOM | 407 | OD1 | ASN | 52 | 72.529 | 39.322 | 33.102 | 1.00 | 34.18 |
| ATOM | 408 | ND2 | ASN | 52 | 72.576 | 37.151 | 32.525 | 1.00 | 34.18 |
| ATOM | 411 | C | ASN | 52 | 73.653 | 38.278 | 37.098 | 1.00 | 34.69 |
| ATOM | 412 | O | ASN | 52 | 73.720 | 37.260 | 37.786 | 1.00 | 34.18 |
| ATOM | 413 | N | LEU | 53 | 73.386 | 39.480 | 37.593 | 1.00 | 28.58 |
| ATOM | 414 | CA | LEU | 53 | 73.156 | 39.733 | 39.008 | 1.00 | 28.58 |
| ATOM | 415 | CB | LEU | 53 | 73.805 | 41.074 | 39.380 | 1.00 | 36.89 |
| ATOM | 416 | CG | LEU | 53 | 73.657 | 41.716 | 40.761 | 1.00 | 36.89 |
| ATOM | 417 | CD1 | LEU | 53 | 74.996 | 42.266 | 41.209 | 1.00 | 36.89 |
| ATOM | 418 | CD2 | LEU | 53 | 72.624 | 42.829 | 40.711 | 1.00 | 36.89 |
| ATOM | 419 | C | LEU | 53 | 71.649 | 39.753 | 39.266 | 1.00 | 28.58 |
| ATOM | 420 | O | LEU | 53 | 70.876 | 40.229 | 38.432 | 1.00 | 36.89 |
| ATOM | 421 | N | ALA | 54 | 71.233 | 39.208 | 40.406 | 1.00 | 17.93 |
| ATOM | 422 | CA | ALA | 54 | 69.817 | 39.157 | 40.763 | 1.00 | 17.93 |
| ATOM | 423 | CB | ALA | 54 | 69.579 | 38.092 | 41.823 | 1.00 | 27.99 |
| ATOM | 424 | C | ALA | 54 | 69.307 | 40.507 | 41.248 | 1.00 | 17.93 |
| ATOM | 425 | O | ALA | 54 | 70.083 | 41.433 | 41.459 | 1.00 | 27.99 |
| ATOM | 426 | N | SER | 55 | 67.996 | 40.617 | 41.417 | 1.00 | 46.64 |
| ATOM | 427 | CA | SER | 55 | 67.390 | 41.857 | 41.881 | 1.00 | 46.64 |
| ATOM | 428 | CB | SER | 55 | 65.914 | 41.917 | 41.473 | 1.00 | 60.10 |
| ATOM | 429 | OG | SER | 55 | 65.769 | 41.953 | 40.062 | 1.00 | 60.10 |
| ATOM | 430 | C | SER | 55 | 67.513 | 41.947 | 43.396 | 1.00 | 46.64 |
| ATOM | 431 | O | SER | 55 | 67.249 | 40.973 | 44.104 | 1.00 | 60.10 |

TABLE 14
Three dimensional coordinates of
LC - CDR3 (GLN88-THR96) from SB249417

| | | | | | | | | | |
|---|---|---|---|---|---|---|---|---|---|
| ATOM | 677 | N | GLN | 88 | 76.228 | 26.138 | 40.949 | 1.00 | 23.98 |
| ATOM | 678 | CA | GLN | 88 | 75.808 | 24.954 | 40.213 | 1.00 | 23.98 |
| ATOM | 679 | CB | GLN | 88 | 74.400 | 24.510 | 40.616 | 1.00 | 33.56 |
| ATOM | 680 | CG | GLN | 88 | 73.285 | 25.370 | 40.066 | 1.00 | 33.56 |
| ATOM | 681 | CD | GLN | 88 | 71.932 | 24.738 | 40.259 | 1.00 | 33.56 |
| ATOM | 682 | OE1 | GLN | 88 | 71.415 | 24.691 | 41.369 | 1.00 | 33.56 |
| ATOM | 683 | NE2 | GLN | 88 | 71.346 | 24.251 | 39.179 | 1.00 | 33.56 |
| ATOM | 686 | C | GLN | 88 | 75.850 | 25.282 | 38.730 | 1.00 | 23.98 |
| ATOM | 687 | O | GLN | 88 | 75.909 | 26.452 | 38.349 | 1.00 | 33.56 |
| ATOM | 688 | N | GLN | 89 | 75.775 | 24.254 | 37.897 | 1.00 | 53.56 |
| ATOM | 689 | CA | GLN | 89 | 75.833 | 24.439 | 36.456 | 1.00 | 53.56 |
| ATOM | 690 | CB | GLN | 89 | 77.082 | 23.752 | 35.888 | 1.00 | 50.20 |
| ATOM | 691 | CG | GLN | 89 | 77.610 | 22.557 | 36.694 | 1.00 | 50.20 |
| ATOM | 692 | CD | GLN | 89 | 76.615 | 21.414 | 36.823 | 1.00 | 50.20 |
| ATOM | 693 | OE1 | GLN | 89 | 75.598 | 21.532 | 37.510 | 1.00 | 50.20 |
| ATOM | 694 | NE2 | GLN | 89 | 76.923 | 20.289 | 36.194 | 1.00 | 50.20 |
| ATOM | 697 | C | GLN | 89 | 74.596 | 23.944 | 35.728 | 1.00 | 53.55 |
| ATOM | 698 | O | GLN | 89 | 73.772 | 23.224 | 36.298 | 1.00 | 50.20 |
| ATOM | 699 | N | TRP | 90 | 74.447 | 24.383 | 34.481 | 1.00 | 42.71 |
| ATOM | 700 | CA | TRP | 90 | 73.331 | 23.967 | 33.641 | 1.00 | 42.71 |
| ATOM | 701 | CB | TRP | 90 | 73.336 | 24.762 | 32.327 | 1.00 | 107.30 |
| ATOM | 702 | CG | TRP | 90 | 72.630 | 24.093 | 31.185 | 1.00 | 107.30 |
| ATOM | 703 | CD2 | TRP | 90 | 73.219 | 23.652 | 29.955 | 1.00 | 107.30 |
| ATOM | 704 | CE2 | TRP | 90 | 72.197 | 23.037 | 29.200 | 1.00 | 107.30 |
| ATOM | 705 | CE3 | TRP | 90 | 74.513 | 23.715 | 29.418 | 1.00 | 107.30 |
| ATOM | 706 | CD1 | TRP | 90 | 71.313 | 23.748 | 31.124 | 1.00 | 107.30 |
| ATOM | 707 | NE1 | TRP | 90 | 71.044 | 23.111 | 29.935 | 1.00 | 107.30 |
| ATOM | 708 | CZ2 | TRP | 90 | 72.427 | 22.485 | 27.935 | 1.00 | 107.30 |
| ATOM | 709 | CZ3 | TRP | 90 | 74.742 | 23.165 | 28.157 | 1.00 | 107.30 |
| ATOM | 710 | CH2 | TRP | 90 | 73.702 | 22.559 | 27.432 | 1.00 | 107.30 |
| ATOM | 711 | C | TRP | 90 | 73.497 | 22.473 | 33.371 | 1.00 | 42.71 |
| ATOM | 712 | O | TRP | 90 | 72.522 | 21.720 | 33.366 | 1.00 | 107.30 |
| ATOM | 713 | N | SER | 91 | 74.746 | 22.068 | 33.154 | 1.00 | 53.84 |
| ATOM | 714 | CA | SER | 91 | 75.130 | 20.683 | 32.897 | 1.00 | 53.84 |
| ATOM | 715 | CB | SER | 91 | 74.815 | 19.789 | 34.106 | 1.00 | 38.65 |
| ATOM | 716 | OG | SER | 91 | 73.457 | 19.379 | 34.150 | 1.00 | 38.65 |
| ATOM | 717 | C | SER | 91 | 74.545 | 20.057 | 31.639 | 1.00 | 53.84 |
| ATOM | 718 | O | SER | 91 | 73.464 | 20.425 | 31.184 | 1.00 | 38.65 |
| ATOM | 719 | N | ILE | 92 | 75.313 | 19.148 | 31.051 | 1.00 | 51.50 |
| ATOM | 720 | CA | ILE | 92 | 74.874 | 18.421 | 29.867 | 1.00 | 51.50 |
| ATOM | 721 | CB | ILE | 92 | 76.070 | 17.967 | 28.991 | 1.00 | 66.93 |
| ATOM | 722 | CG2 | ILE | 92 | 75.598 | 17.678 | 27.570 | 1.00 | 66.93 |
| ATOM | 723 | CG1 | ILE | 92 | 77.154 | 19.047 | 28.948 | 1.00 | 66.93 |
| ATOM | 724 | CD1 | ILE | 92 | 78.444 | 18.594 | 28.271 | 1.00 | 66.93 |
| ATOM | 725 | C | ILE | 92 | 74.211 | 17.171 | 30.446 | 1.00 | 51.50 |
| ATOM | 726 | O | ILE | 92 | 73.268 | 16.621 | 29.881 | 1.00 | 66.93 |
| ATOM | 727 | N | ASN | 93 | 74.714 | 16.755 | 31.605 | 1.00 | 60.16 |
| ATOM | 728 | CA | ASN | 93 | 74.232 | 15.580 | 32.319 | 1.00 | 60.16 |
| ATOM | 729 | CB | ASN | 93 | 75.290 | 14.473 | 32.228 | 1.00 | 59.88 |
| ATOM | 730 | CG | ASN | 93 | 74.696 | 13.083 | 32.312 | 1.00 | 59.88 |
| ATOM | 731 | OD1 | ASN | 93 | 74.187 | 12.673 | 33.354 | 1.00 | 59.88 |
| ATOM | 732 | ND2 | ASN | 93 | 74.769 | 12.345 | 31.213 | 1.00 | 59.88 |
| ATOM | 735 | C | ASN | 93 | 74.046 | 16.023 | 33.775 | 1.00 | 60.16 |
| ATOM | 736 | O | ASN | 93 | 74.952 | 16.620 | 34.363 | 1.00 | 59.88 |
| ATOM | 737 | N | PRO | 94 | 72.883 | 15.709 | 34.377 | 1.00 | 45.30 |
| ATOM | 738 | CD | PRO | 94 | 72.057 | 14.607 | 33.849 | 1.00 | 61.85 |
| ATOM | 739 | CA | PRO | 94 | 72.443 | 16.011 | 35.740 | 1.00 | 45.30 |
| ATOM | 740 | CB | PRO | 94 | 72.227 | 14.621 | 36.314 | 1.00 | 61.85 |
| ATOM | 741 | CG | PRO | 94 | 71.516 | 13.930 | 35.137 | 1.00 | 61.85 |
| ATOM | 742 | C | PRO | 94 | 73.280 | 16.922 | 36.644 | 1.00 | 45.30 |
| ATOM | 743 | O | PRO | 94 | 74.461 | 16.670 | 36.913 | 1.00 | 61.85 |
| ATOM | 744 | N | ARG | 95 | 72.607 | 17.959 | 37.141 | 1.00 | 55.22 |
| ATOM | 745 | CA | ARG | 95 | 73.181 | 18.975 | 38.024 | 1.00 | 55.22 |
| ATOM | 746 | CB | ARG | 95 | 72.097 | 19.992 | 38.393 | 1.00 | 42.76 |
| ATOM | 747 | CG | ARG | 95 | 71.364 | 20.575 | 37.194 | 1.00 | 42.76 |
| ATOM | 748 | CD | ARG | 95 | 70.022 | 21.178 | 37.591 | 1.00 | 42.76 |
| ATOM | 749 | NE | ARG | 95 | 68.909 | 20.524 | 36.902 | 1.00 | 42.76 |
| ATOM | 750 | CZ | ARG | 95 | 67.634 | 20.899 | 36.995 | 1.00 | 42.76 |
| ATOM | 751 | NH1 | ARG | 95 | 67.286 | 21.931 | 37.752 | 1.00 | 42.76 |
| ATOM | 754 | NH2 | ARG | 95 | 66.701 | 20.246 | 36.315 | 1.00 | 42.76 |
| ATOM | 757 | C | ARG | 95 | 73.753 | 18.352 | 39.294 | 1.00 | 55.22 |
| ATOM | 758 | O | ARG | 95 | 73.351 | 17.255 | 39.684 | 1.00 | 42.76 |
| ATOM | 759 | N | THR | 96 | 74.657 | 19.066 | 39.963 | 1.00 | 47.74 |
| ATOM | 760 | CA | THR | 96 | 75.270 | 18.543 | 41.183 | 1.00 | 47.74 |
| ATOM | 761 | CB | THR | 96 | 76.630 | 17.877 | 40.875 | 1.00 | 32.33 |
| ATOM | 762 | OG1 | THR | 96 | 77.370 | 18.701 | 39.967 | 1.00 | 32.33 |
| ATOM | 763 | CG2 | THR | 96 | 76.433 | 16.494 | 40.264 | 1.00 | 32.33 |
| ATOM | 764 | C | THR | 96 | 75.456 | 19.495 | 42.370 | 1.00 | 47.74 |
| ATOM | 765 | O | THR | 96 | 75.379 | 19.052 | 43.515 | 1.00 | 32.33 |

### REFERENCES

Brünger A. T., (1990) Acta Cryst. A46:46-57.
Brüneer A. T., (1991) Acta Cryst. A47: 195-204.
Brünger A. T., X-PLOR 3.0 Manual (Yale University, 1992).
Huber R., (1985). In Molecular Replacement. Proc. of the Daresbury Study Weekend, Daresbury, February 1985, pp. 58-61. SERC Daresbury Laboratory, Daresbury, Warrington, England.
Monfardini C., Kieber-Emmons T. Voet D., Godillot A. P., Weiner D. B., Williams W. V., (1996) J. Biol. Chem. 271:2966-2971.
Otwinowski Z., “Oscillation Data Reduction,” in Proceedings of the CCP4 Study Weekend: “Data Collection and Processing,” 29-30 Jan. 1993, comiled by L. Sawyer, N. Isaacs and S. Bailey (SERC Daresbury Laboratory, England), pp. 56-62.
Rossmann M. G. and Blow D. M., (1962). Acta Cryst. 15:24-31.
Rusinko, A., (1993) Chem. Des. Auto. News 8:44-47.
Zhao B., Helms L. R., DesJarlais R. L., Abdel-Meguid S. S. & Wetzel R., (1995) Nature Struct. Biol. 2: 1131-1137.

The present invention may be embodied in other specific forms without departing from the spirit or essential attributes thereof, and, accordingly, reference should be made to the appended claims, rather than to the foregoing specification, as indicating the scope of the invention.

1. A BC2 Fab fragment crystal.

2. A Fab fragment crystal containing BC2 complementarity determining regions (CDRs).

3. The crystal of claim 2 wherein the CDRs are characterized by the coordinates of Tables 3-8.

4. A SB249417 Fab fragment crystal.

5. The crystal of claim 4 wherein the CDRs are characterized by the coordinates of Tables 9-14.

6. A method for identifying a peptidomimetic having Factor IX binding activity comprising:
a. searching a small molecule structural database with CDR structural parameters derived from the crystal of claim 1, 2 or 4;
b. selecting a molecular structure from the database which mimics the CDR structural parameters;
c. synthesizing the selected molecular structure; and
d. screening the synthesized molecule for Factor IX binding activity.

7. The method of claim 6 wherein the synthesized molecule is further screened for antithrombotic activity.

8. The method of claim 7 wherein the synthesized molecule is further screened for self-limiting, neutralizing activity.

9. The method of claim 6 wherein the selected molecular structure mimics the parameters of CDR residues HC-Asn35, HC-Trp50, and LC-Arg95.

10. A computer-readable medium having BC2 CDR structural information stored thereon.

11. A computer-readable medium having SB249417 CDR structural information stored thereon.
